(19) United States
(12) Patent Application Publication
(10) Pub. No.: US 2010/0124542 A1
(43) Pub. Date: May 20, 2010

(54) AMINE DENDRIMERS

(75) Inventors: Pradeep K. Dhal, Westford, MA (US); Stephen Randall Holmes-Farley, Arlington, MA (US); Chad C. Huval, Somerville, MA (US)

Correspondence Address: JONES DAY, 222 EAST 41ST ST, NEW YORK, NY, 10017, US

(73) Assignee: Genzyme Corporation, Cambridge, MA (US)

(21) Appl. No.: 12/309,414

(22) PCT Filed: Jul. 16, 2007
(86) PCT No.: PCT/US07/16240

§ 371 Date: Dec. 2, 2009

Related U.S. Application Data
(60) Provisional application No. 60/831,461, filed on Jul. 18, 2006.
(60) Provisional application No. 60/837,322, filed on Aug. 14, 2006.
(60) Provisional application No. 60/847,641, filed on Sep. 28, 2006.

(51) Int. Cl. A61K 31/765; C08G 65/26; A61P 1/00

(52) U.S. Cl. 424/78.37; 528/405

(57) ABSTRACT

Ion binding compounds and compositions may include compounds, polymers and compositions that include amine moieties. Ion binding polymers may be crosslinked amine polymers and may be used to remove ions, such as phosphate ions, from the gastrointestinal tract of animals, such as humans. Such compounds, polymers and compositions may be used therapeutically to treat a variety of medical conditions, such as hyperphosphatemia.

### FIELD OF THE INVENTION

This invention relates to polymeric substances for binding target ions, and more specifically relates to pharmaceutically acceptable amine compounds, polymers and compositions for binding target ions.

### BACKGROUND OF THE INVENTION

Hyperphosphatemia frequently accompanies diseases associated with inadequate renal function such as end stage renal disease (ESRD), hyperparathyroidism, and certain other medical conditions. The condition, especially if present over extended periods of time, leads to severe abnormalities in calcium and phosphorus metabolism and can be manifested by aberrant calcification in joints, lungs, and eyes.

Therapeutic efforts to reduce serum phosphate include dialysis, reduction in dietary phosphate, and oral administration of insoluble phosphate binders to reduce gastrointestinal absorption. Many such treatments have a variety of unwanted side effects and/or have less than optimal phosphate binding properties, including potency and efficacy. Accordingly, there is a need for compositions and treatments with good phosphate-binding properties and good side effect profiles.

### BRIEF SUMMARY OF THE INVENTION

In one first aspect, the present invention relates to compounds, polymers and compositions comprising amine moieties which may be crosslinked. The polymers can be crosslinked amine polymers. The compositions can comprise one or more crosslinked amine polymers. Several embodiments of the invention, including this aspect of the invention, are described in further detail as follows. Generally, each of these embodiments can be used in various and specific combinations, and with other aspects and embodiments unless otherwise stated herein.

In addition to the compounds and polymers of the present invention as described herein, other forms of the compounds and polymers are within the scope of the invention including pharmaceutically acceptable salts, solvates, hydrates, prodrugs, polymorphs, clathrates, and isotopic variants and mixtures thereof of the compounds and/or polymers.

In addition, compounds and polymers of the invention may have optical centers, chiral centers or double bonds and the amine compounds and amine polymers of the present invention include all of the isomeric forms of these compounds and polymers, including optically pure forms, racemates, diastereomers; enantiomers, tautomers and/or mixtures thereof.

In a first embodiment, the invention is, consists essentially of, or comprises a crosslinked amine polymer that includes or is derived from an amine compound represented by Formula I or a residue thereof, as follows:

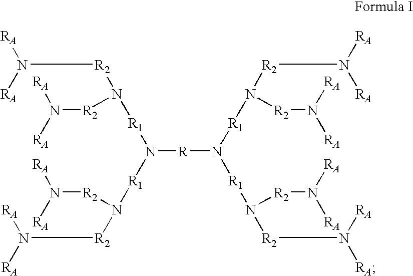

wherein
R independently represents:

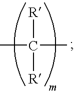

R₁ independently represents:

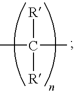

R₂ independently represents:

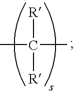

R_{A} independently represents:

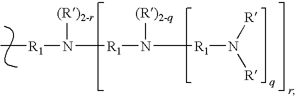

and where m independently represents an integer from 1 to 20, for example, 1-15, 1-2, 3-6, 7-10, 11-15, such as 1, 2, 3, 4, 5, 6, 7, 8, 9, 10, 11, 12, 13, 14, 15, 16, 17, 18, 19 or 20; n and s independently represent an integer from 1-20, for example, 1-15, 1-2, 3-5, 6-10, 11-15, such as 2, 3, 4, 5, or 6; q and r independently represent an integer from 0-2, for example 0, 1 or 2; and R′ independently represents a hydrogen radical; or a substituted or un-substituted alkyl radical; or a substituted or un-substituted aryl radical; or R′ and a neighboring R′ together represent a link or links comprising a residue of a crosslinking agent, for example epichlorohydrin or other crosslinking agents, a substituted or un-substituted alicyclic radical, a substituted or un-substituted aromatic radical, or a substituted or un-substituted heterocyclic radical; or R′ represents a link with another compound, for example, another amine compound or residue thereof, another polymeric compound or residue thereof, or a crosslinking compound or residue thereof.

In another aspect, the invention provides methods of treating an animal, including a human. The method generally involves administering an effective amount of a crosslinked amine polymer described herein.

Another aspect of the invention is a pharmaceutical composition comprising one or more polymers of the present invention with at least one pharmaceutically acceptable carrier. The polymers described herein have several therapeutic applications. For example, the crosslinked amine polymers are useful in removing ions, for example phosphate, from the gastrointestinal tract. In some embodiments, the crosslinked amine polymers are used in the treatment of phosphate imbalance disorders and renal diseases.

In yet another aspect, the crosslinked amine polymers are useful for removing other anionic solutes, such as chloride, bicarbonate, and/or oxalate ions. Polymers removing oxalate ions find use in the treatment of oxalate imbalance disorders. Polymers removing chloride ions find use in treating acidosis, for example. In some embodiments, the crosslinked amine polymers are useful for removing bile acids and related compounds.

The invention further provides compositions containing any of the above polymers where the polymer is in the form of particles and where the polymeric particles are encased in an outer shell.

In another aspect, the invention provides pharmaceutical compositions. In one embodiment, the pharmaceutical composition contains a crosslinked amine compound of the invention and a pharmaceutically acceptable excipient. In some embodiments, the composition is a liquid formulation in which the polymer is dispersed in a liquid vehicle of water and suitable excipients. In some embodiments, the invention provides a pharmaceutical composition comprising the polymer for binding a target ion, and one or more suitable pharmaceutical excipients, where the composition is in the form of a tablet, sachet, slurry, food formulation, troche, capsule, elixir, suspension, syrup, wafer, chewing gum or lozenge. In some embodiments the composition contains a pharmaceutical excipient selected from the group consisting of sucrose, mannitol, xylitol, maltodextrin, fructose, sorbitol, and combinations thereof. In some embodiments the target anion of the polymer is phosphate. In some embodiments the polymer is more than about 50% of the weight of the tablet. In some embodiments, the tablet is of cylindrical shape with a diameter of from about 12 mm to about 28 mm and a height of from about 1 mm to about 8 mm and the polymer comprises more than 0.6 to about 2.0 gm of the total weight of the tablet. In some of the compositions of the invention, the excipients are chosen from the group consisting of sweetening agents, binders, lubricants, and disintegrants. Optionally, the polymer is present as particles of less than about 80 μm mean diameter. In some of these embodiments, the sweetening agent is selected from the group consisting of sucrose, mannitol, xylitol, maltodextrin, fructose, and sorbitol, and combinations thereof.

### DETAILED DESCRIPTION OF THE INVENTION

### Amine Polymers

In one aspect, the present invention provides compounds, compositions and methods of using compounds or compositions comprising a polymer that includes an amine compound or residue thereof according to Formula I. In some embodiments, the amine compound may be crosslinked. In some embodiments, compounds may comprise polymers that may be homopolymers or copolymers including, for example, copolymers comprising or derived from two or more of the amine compounds described herein.

In addition, some embodiments may include multiple amine compounds that repeat in a copolymer or polymer. Such polymers may include one or more additional compounds that may be included in the polymer backbone or as pendant groups either individually or as repeating groups, and that may provide separation between the individual amine compounds.

As used herein, unless otherwise stated, the term “derived from” is understood to mean: produced or obtained from another substance by chemical reaction, especially directly derived from the reactants, for example amine compound reacted with a crosslinking agent results in a polymer that is derived from the amine compound.

In some embodiments, the invention is a compound or composition or method for removing phosphate from the gastrointestinal tract of an animal by administering an effective amount of a polymer that includes or is derived from an amine compound represented by Formula I or a residue thereof, as follows:

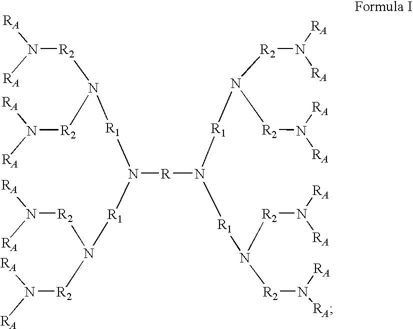

wherein
R independently represents:

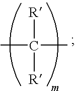

R₁ independently represents:

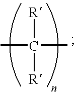

R₂ independently represents:

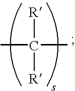

R_{A} independently represents:

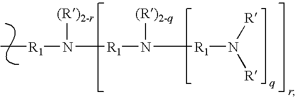

and where m independently represents an integer from 1 to 20, for example, 1-15, 1-2, 3-6, 7-10, 11-15, such as 1, 2, 3, 4, 5, 6, 7, 8, 9, 10, 11, 12, 13, 14, 15, 16, 17, 18, 19 or 20; n and s independently represent an integer from 1-20, for example, 1-15, 1-2, 3-5, 6-10, 11-15, such as 2, 3, 4, 5, or 6; q and r independently represent an integer from 0-2, for example 0, 1 or 2; and R′ independently represents a hydrogen radical; or a substituted or un-substituted alkyl radical; or a substituted or un-substituted aryl radical; or R′ and a neighboring R′ together represent a link or links comprising a residue of a crosslinking agent, for example epichlorohydrin or other crosslinking agents, a substituted or un-substituted alicyclic radical, a substituted or un-substituted aromatic radical, or a substituted or un-substituted heterocyclic radical; or R′ represents a link with another compound.

In some embodiments, the invention is a compound or composition or method for removing phosphate from the gastrointestinal tract of an animal by administering an effective amount of a polymer that includes or is derived from an amine compound represented by Formula II or a residue thereof, as follows:

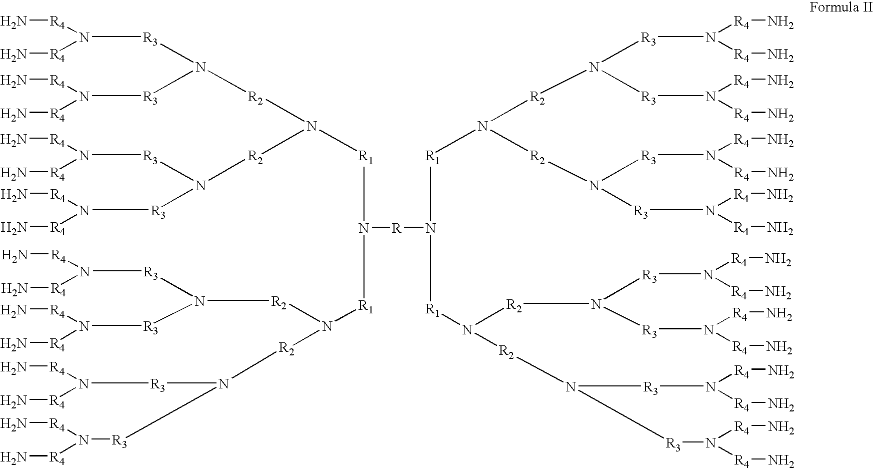

wherein
R independently represents:

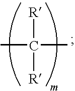

R₁ independently represents:

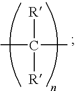

R₂ independently represents:

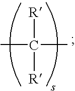

R₃ independently represents:

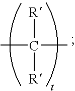

R₄ independently represents:

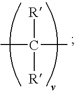

and where m independently represents an integer from 1 to 20, for example, 1-15, 1-2, 3-6, 7-10, 11-15, such as 1, 2, 3, 4, 5, 6, 7, 8, 9, 10, 11, 12, 13, 14, 15, 16, 17, 18, 19 or 20; n, s, t and v independently represent an integer from 1-20, for example, 1-15, 1-2, 3-5, 6-10, 11-15, such as 2, 3, 4, 5, or 6; and R′ independently represents a hydrogen radical; or a substituted or un-substituted alkyl radical; or a substituted or un-substituted aryl radical; or R′ and a neighboring R′ together represent a link or links comprising a residue of a crosslinking agent, for example epichlorohydrin or other crosslinking agents, a substituted or un-substituted alicyclic radical, a substituted or un-substituted aromatic radical, or a substituted or un-substituted heterocyclic radical; or R′ represents a link with another compound.

In some embodiments, the invention is a compound or composition or method for removing phosphate from the gastrointestinal tract of an animal by administering an effective amount of a polymer that includes or is derived from an amine compound represented by Formula III or a residue thereof, as follows:

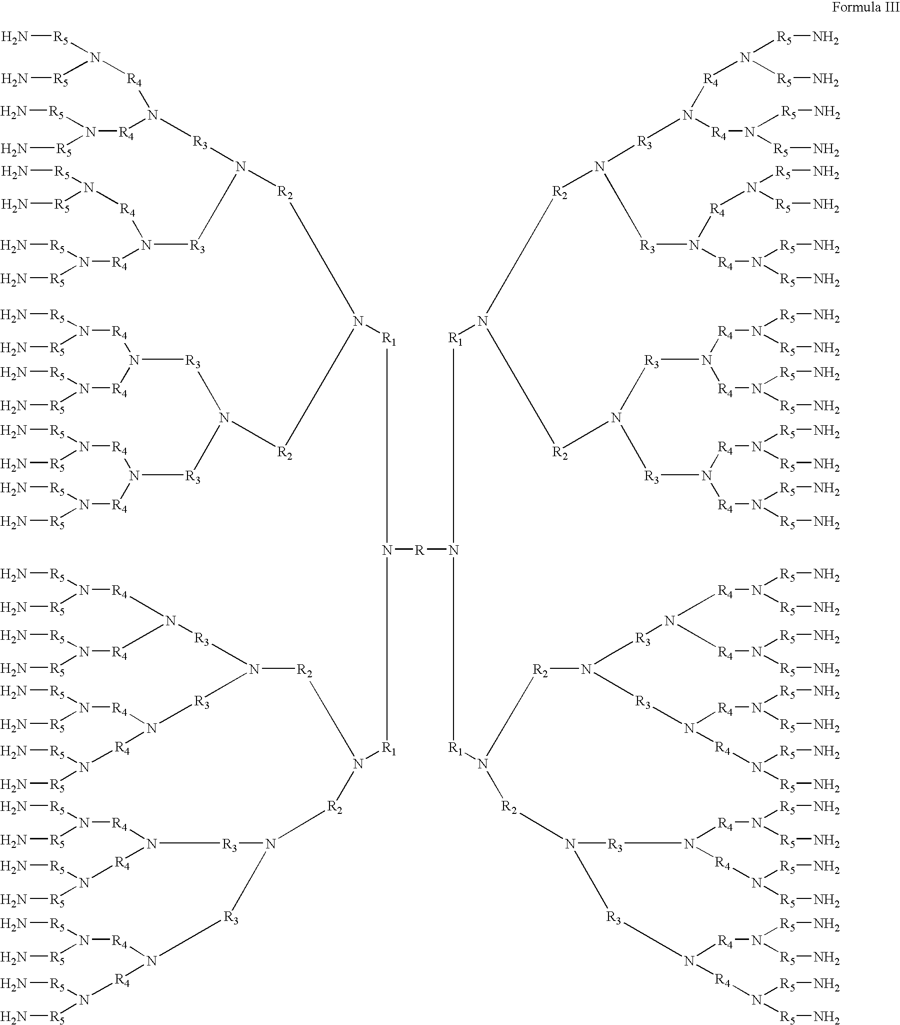

wherein
R independently represents:

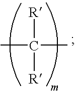

R₁ independently represents:

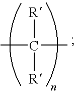

R₂ independently represents:

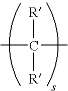

R₃ independently represents:

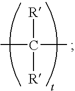

R₄ independently represents:

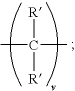

R₅ independently represents:

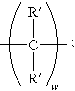

and where m independently represents an integer from 1 to 20, for example, 1-15, 1-2, 3-6, 7-10, 11-15, such as 1, 2, 3, 4, 5, 6, 7, 8, 9, 10, 11, 12, 13, 14, 15, 16, 17, 18, 19 or 20; n, s, t, v and w independently represent an integer from 1-20, for example, 1-15, 1-2, 3-5, 6-10, 11-15, such as 2, 3, 4, 5, or 6; and R′ independently represents a hydrogen radical; or a substituted or un-substituted alkyl radical; or a substituted or un-substituted aryl radical; or R′ and a neighboring R′ together represent a link or links comprising a residue of a crosslinking agent, for example epichlorohydrin or other crosslinking agents, a substituted or un-substituted alicyclic radical, a substituted or un-substituted aromatic radical, or a substituted or un-substituted heterocyclic radical; or R′ represents a link with another compound.

In some embodiments, the amine compound may be represented by the following Formula IV or a residue thereof:

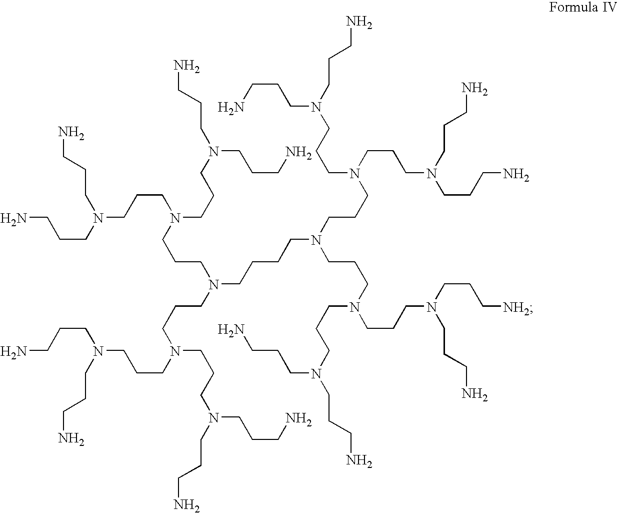

In some embodiments, the invention is a compound or composition or method for removing phosphate from the gastrointestinal tract of an animal by administering an effective amount of a polymer that includes or is derived from an amine compound represented by Formula V or a residue thereof, as follows:

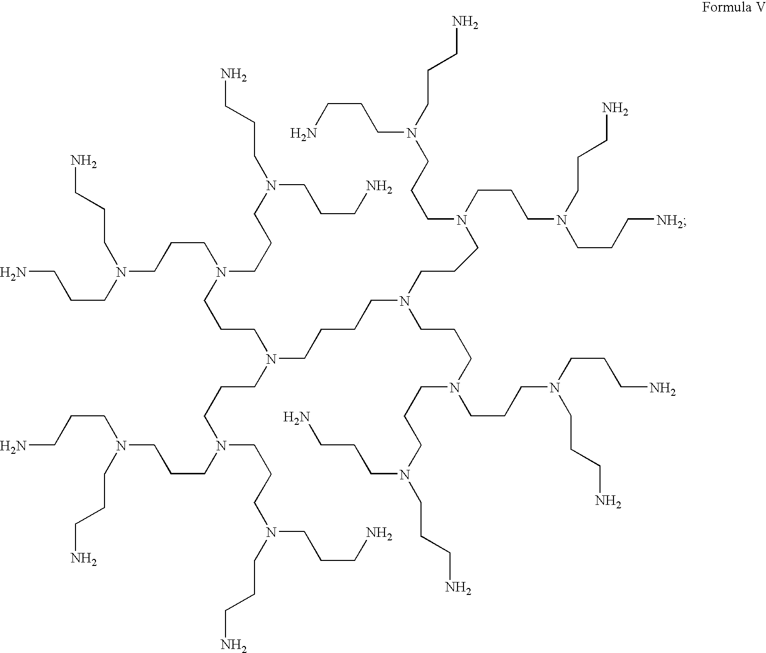

In some embodiments, the invention is a compound or composition or method for removing phosphate from the gastrointestinal tract of an animal by administering an effective amount of a polymer that includes or is derived from an amine compound represented by Formula VI or a residue thereof, as follows:

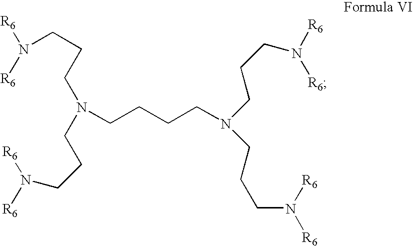

wherein
R₆, independently represents:

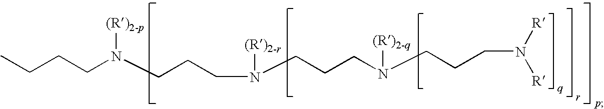

where p, q and r independently represent an integer from 0-2, for example 0, 1 or 2; and R′ independently represents a hydrogen radical; or a substituted or un-substituted alkyl radical; or R′ and a neighboring R′ together represent a link or links comprising a residue of a crosslinking agent, for example epichlorohydrin or other crosslinking agents; or R′ represents a link with another compound.

In some embodiments, the invention is a compound or composition or method for removing phosphate from the gastrointestinal tract of an animal by administering an effective amount of a polymer that includes or is derived from an amine compound represented by Formula VII or a residue thereof, as follows:

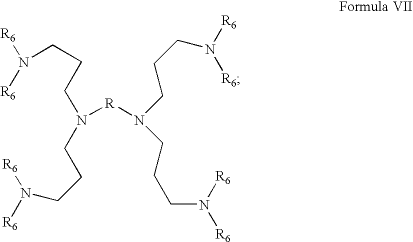

wherein
R independently represents:

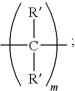

R₆ independently represents:

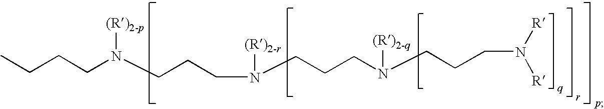

wherein m independently represents an integer from 1 to 8, for example, 1-2, 2-6, 6-8, such as 1, 2, 3, 4, 5, 6, 7, or 8; p, q and r independently represent an integer from 0-2, for example 0, 1 or 2; and R′ independently represents a hydrogen radical; or a substituted or un-substituted alkyl radical; or R′ and a neighboring R′ together represent a link or links comprising a residue of a crosslinking agent, for example epichlorohydrin or other crosslinking agents; or R′ represents a link with another compound.

In some embodiments, the invention is a compound or composition or method for removing phosphate from the gastrointestinal tract of an animal by administering an effective amount of a polymer that includes or is derived from an amine compound represented by Formula VIII or a residue thereof, as follows:

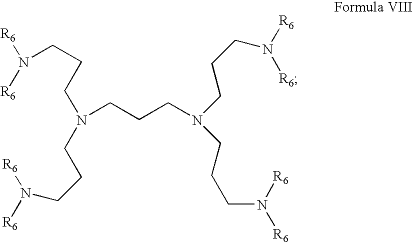

wherein
R₆ independently represents:

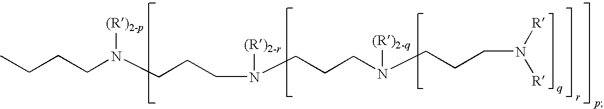

wherein p, q and r independently represent an integer from 0-2, for example 0, 1 or 2; and R′ independently represents a hydrogen radical; or a substituted or un-substituted alkyl radical; or R′ and a neighboring R′ together represent a link or links comprising a residue of a crosslinking agent, for example epichlorohydrin or other crosslinking agents; or R′ represents a link with another compound.

In some embodiments, the invention is a compound or composition or method for removing phosphate from the gastrointestinal tract of an animal by administering an effective amount of a polymer that includes or is derived from an amine compound represented by Formula IX or a residue thereof, as follows:

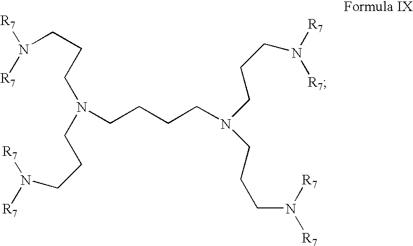

wherein
R₇ independently represents:

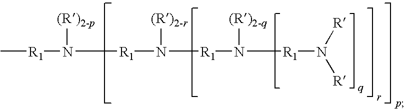

R₁ independently represents:

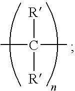

wherein n independently represents an integer from 1-6, for example, 2-6, 1-2, or 3-5, such as 1, 2, 3, 4, 5, or 6; p, q and r independently represent an integer from 0-2, for example 0, 1 or 2; and R′ independently represents a hydrogen radical; or a substituted or un-substituted alkyl radical; or R′ and a neighboring R′ together represent a link or links comprising a residue of a crosslinking agent, for example epichlorohydrin or other crosslinking agents; or R′ represents a link with another compound.

In some embodiments, the invention is a compound or composition or method for removing phosphate from the gastrointestinal tract of an animal by administering an effective amount of a polymer that includes or is derived from an amine compound represented by Formula X or a residue thereof, as follows:

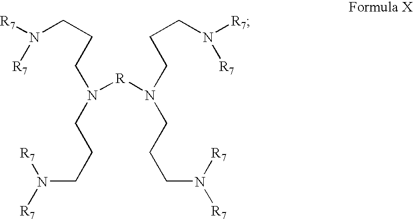

wherein
R₇ independently represents:

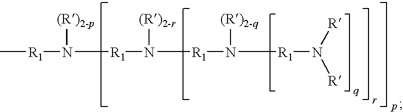

R independently represents:

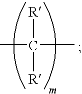

R₁ independently represents:

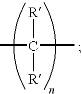

wherein m independently represents an integer from 1 to 8, for example, 1-2, 2-6, 6-8, such as 1, 2, 3, 4, 5, 6, 7, or 8; n independently represents an integer from 1-6, for example, 2-6, 1-2, or 3-5, such as 1, 2, 3, 4, 5, or 6; p, q and r independently represent an integer from 0-2, for example 0, 1 or 2; and R′ independently represents a hydrogen radical; or a substituted or un-substituted alkyl radical; or R′ and a neighboring R′ together represent a link or links comprising a residue of a crosslinking agent, for example epichlorohydrin or other crosslinking agents; or R′ represents a link with another compound.

In some embodiments, the invention is a compound or composition or method for removing phosphate from the gastrointestinal tract of an animal by administering an effective amount of a polymer that includes or is derived from an amine compound represented by Formula XI or a residue thereof, as follows:

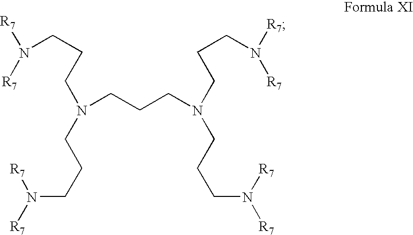

wherein
R₇ independently represents:

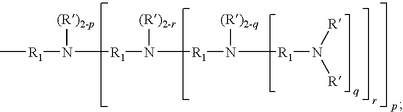

R₁ independently represents:

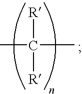

wherein n independently represents an integer from 1-6, for example, 2-6, 1-2, or 3-5, such as 1, 2, 3, 4, 5, or 6; p, q and r independently represent an integer from 0-2, for example 0, 1 or 2; and R′ independently represents a hydrogen radical; or a substituted or un-substituted alkyl radical; or R′ and a neighboring R′ together represent a link or links comprising a residue of a crosslinking agent, for example epichlorohydrin or other crosslinking agents; or R′ represents a link with another compound.

In one embodiment, the amine compound may be represented by the following Formula XII or a residue thereof:

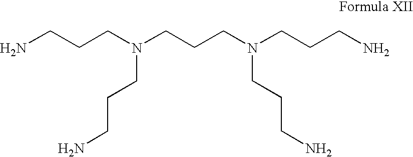

In some embodiments, examples of suitable amine compounds may be: 4,25-bis(3-aminopropyl)-12,17-[3-[bis[3-[bis(3-aminopropyl)amino]propyl]amino]propyl]-8,21-bis[3-[bis(3-aminopropyl)amino]propyl]-4,8,12,17,21,25-hexaazaoctacosane-1,28-diamine; 1,4-bis[bis[3-[bis[3-[bis(3-aminopropyl)amino]propyl]amino]propyl]amino]butane; 4,33-bis(3-aminopropyl)-8,29-bis[3-[bis(3-aminopropyl)amino]propyl]-12,25-bis[3-[bis[3-[bis(3-aminopropyl)amino]propyl]amino]propyl]-16,21-bis[3-[bis[3-[bis[3-[bis(3-aminopropyl)amino]propyl]amino]propyl]amino]propyl]-4,8,12,16,21,25,29,33-octaazahexatriacontane-1,36-diamine; 1,4-bis[bis[3-[bis[3-[bis[3-[bis(3-aminopropyl)amino]propyl]amino]propyl]amino]propyl]amino]butane; or 1,4-bis[bis[3-[bis[3-[bis[3-[bis[3-[bis(3-aminopropyl)amino]propyl]amino]propyl]amino]propyl]amino]propyl]amino]butane.

In one embodiment an example of a suitable amine compound may be N,N,N′,N′-tetrakis(3-aminopropyl)-1,3-propanediamine. In another embodiment a suitable amine compound may be an amidoethylethanolamine dendrimer with a 1,4 diaminobutane core inclusive of dendrimer generations 1-6.

In some embodiments, the amine compound is a mixture of more than one amine compound, for example 2-20 such as 2, 3, 4, 5, 6, 7, 8, 9 or 10 amine compounds represented by Formulas I-XII. In some embodiments, the mixture predominantly comprises an amine compound represented by one of Formulas I-XII where p, q and r are independently 0 or 2. For example, in some embodiments a plurality of the mixture, such as greater than 30 wt. %, greater than 40 wt. %, greater than 50 wt. %, greater than 60 wt. % or greater than 70 wt. % based on the total weight of the mixture, comprises an amine compound or residue thereof represented by one of Formulas I-XII where p, q and r are independently 0 or 2. For example, in some embodiments, the mixture comprises greater than 30 wt %, greater than 40 wt. %, greater than 50 wt. %, greater than 60 wt. % or greater than 70 wt. % of an amine compound or residue thereof represented by Formula IV or Formula V.

In some embodiments, the invention comprises a polymer derived from an amine compound that is a mixture of amine compounds, a pharmaceutical composition comprising such a polymer, or a method of using the same in a therapeutically effective amount to remove a compound or ion, such as a phosphorous-containing compound or a phosphorous-containing ion (e.g. phosphate), from the gastrointestinal tract of an animal.

Other embodiments of the invention include polymers formed with amine compounds or residues thereof as pendant groups on a polymer or polymerized backbone of a polymer. Such polymers may be formed by adding one or more polymerizable groups to one or more amine groups on an amine compound to form an amine monomer and then subsequently polymerizing the polymerizable group to form a polymer comprising an amine compound or residue thereof. A schematic example of such an addition follows [it should be noted in the following that an amine compound designated as “AC” is intended to represent an amine compound or residue thereof, of the invention, with an amine group depicted for purposes of illustrating how a polymerizable group may be added to an amine compound]:

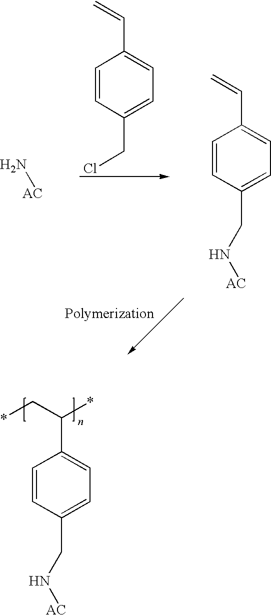

Non-limiting examples of other polymerizable groups that may be used with amine compounds or residues thereof according to embodiments of the invention include:

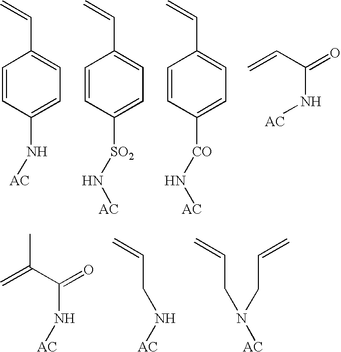

One or more polymerizable groups may be added to each amine compound and thus it is possible to have mixtures of amine monomers having various pendant ACs having differing numbers of polymerizable groups. In addition, the polymers made in this fashion may be modified, crosslinked, formed into a network or substituted post polymerization using techniques known to those of skill in the art. Such modification may be performed for any number of reasons, including to improve efficacy, tolerability or reduce side effects.

Amine monomers may also be formed by addition of amine compounds to amine-reactive polymers by reacting one or more amine groups of the amine monomers with one or amine-reactive groups on the amine-reactive polymers. Examples of some amine reactive polymers include:

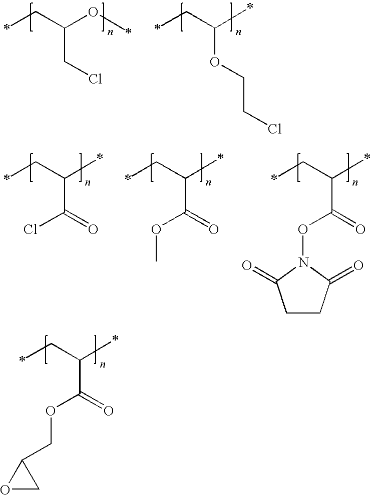

The amine compounds or amine monomers may also serve as multifunctional amine monomers to form polymers. For example, when the amine compounds or the polymers formed from the amine monomers are crosslinked, the crosslinking reaction may be carried out either in solution of bulk (i.e. using the neat amine and neat crosslinking agents) or in dispersed media. When a bulk process is used, solvents are selected so that they co-dissolve the reactants and do not interfere with the crosslinking reaction. Suitable solvents include water, low boiling alcohols (methanol, ethanol, butanol), dimethylformamide, dimethylsulfoxide, acetone, methylethylketone, and the like.

Other polymerization methods may include a single polymerization reaction, stepwise addition of individual monomers via a series of reactions, the stepwise addition of blocks of monomers, combinations of the foregoing, or any other method of polymerization, such as, for example, direct or inverse suspension, condensation, emulsion, precipitation techniques, polymerization in aerosol or using bulk polymerization/crosslinking methods and size reduction processes such as extrusion and grinding. Processes can be carried out as batch, semi-continuous and continuous processes. For processes in dispersed media, the continuous phase can be selected from apolar solvents such as toluene, benzene, hydrocarbon, halogenated solvents, supercritical carbon dioxide, and the like. With a direct suspension process, water can be used, although salt brines are also useful to “salt out” the amine and crosslinking agents in a droplet separate phase.

Polymers of the invention may be copolymerized with one or more other monomers or oligomers or other polymerizable groups, may be crosslinked, may have crosslinking or other linking agents or monomers within the polymer backbone or as pendant groups or may be formed or polymerized to form a network or mixed network of: amine compounds or residues thereof, amine monomers or residues thereof. The network may include multiple connections between the same or different molecules that may be direct or may include one or more linking groups such as crosslinking agents or other monomers or oligomers or residues thereof.

Non-limiting examples of comonomers which may be used alone or in combination include: styrene, substituted styrene, alkyl acrylate, substituted alkyl acrylate, alkyl methacrylate, substituted alkyl methacrylate, acrylonitrile, methacrylonitrile, acrylamide, methacrylamide, N-alkylacrylamide, N-alkylmethacrylamide, N,N-dialkylacrylamide, N,N-dialkylmethacrylamide, isoprene, butadiene, ethylene, vinyl acetate, N-vinyl amide, maleic acid derivatives, vinyl ether, allyle, methallyl monomers and combinations thereof. Functionalized versions of these monomers may also be used. Additional specific monomers or comonomers that may be used in this invention include, but are not limited to, methyl methacrylate, ethyl methacrylate, propyl methacrylate (all isomers), butyl methacrylate (all isomers), 2-ethylhexyl methacrylate, isobornyl methacrylate, methacrylic acid, benzyl methacrylate, phenyl methacrylate, methacrylonitrile, .alpha.-methylstyrene, methyl acrylate, ethyl acrylate, propyl acrylate (all isomers), butyl acrylate (all isomers), 2-ethylhexyl acrylate, isobornyl acrylate, acrylic acid, benzyl acrylate, phenyl acrylate, acrylonitrile, styrene, glycidyl methacrylate, 2-hydroxyethyl methacrylate, hydroxypropyl methacrylate (all isomers), hydroxybutyl methacrylate (all isomers), N,N-dimethylaminoethyl methacrylate, N,N-diethylaminoethyl methacrylate, triethyleneglycol methacrylate, itaconic anhydride, itaconic acid, glycidyl acrylate, 2-hydroxyethyl acrylate, hydroxypropyl acrylate (all isomers), hydroxybutyl acrylate (all isomers), N,N-dimethylaminoethyl acrylate, N,N-diethylaminoethyl acrylate, triethyleneglycol acrylate, methacrylamide, N-methylacrylamide, N,N-dimethylacrylamide, N-tert-butylmethacrylamide, N—N-butylmethacrylamide, N-methylolmethacrylamide, N-ethylolmethacrylamide, N-tert-butylacrylamide, N-n-butylacrylamide, N-methylolacrylamide, N-ethylolacrylamide, 4-acryloylmorpholine, vinyl benzoic acid (all isomers), diethylaminostyrene (all isomers), α-methylvinyl benzoic acid (all isomers), diethylamino α-methylstyrene (all isomers), p-vinylbenzene sulfonic acid, p-vinylbenzene sulfonic sodium salt, trimethoxysilylpropyl methacrylate, triethoxysilylpropyl methacrylate, tributoxysilylpropyl methacrylate, dimethoxymethylsilylpropyl methacrylate, diethoxymethylsilylpropyl methacrylate, dibutoxymethylsilylpropyl methacrylate, diisopropoxymethylsilylpropyl methacrylate, dimethoxysilylpropyl methacrylate, diethoxysilylpropyl methacrylate, dibutoxysilylpropyl methacrylate, diisopropoxysilylpropyl methacrylate, trimethoxysilylpropyl acrylate, triethoxysilylpropyl acrylate, tributoxysilylpropyl acrylate, dimethoxymethylsilylpropyl acrylate, diethoxymethylsilylpropyl acrylate, dibutoxymethylsilylpropyl acrylate, diisopropoxymethylsilylpropyl acrylate, dimethoxysilylpropyl acrylate, diethoxysilylpropyl acrylate, dibutoxysilylpropyl acrylate, diisopropoxysilylpropyl acrylate, maleic anhydride, N-phenylmaleimide, N-butylmaleimide, N-vinylformamide, N-vinyl acetamide, allylamine, methallylamine, allylalcohol, methyl-vinylether, ethylvinylether, butylvinyltether, butadiene, isoprene, chloroprene, ethylene, vinyl acetate and combinations thereof.

In some embodiments, polymers of the invention are crosslinked using crosslinking agents, and may not dissolve in solvents, and, at most, swell in solvents. The swelling ratio is typically in the range of about 1 to about 20; for example 2 to 10, 2.5 to 8, 3 to 6 such as less than 5, less than 6, or less than 7. In some embodiments, the polymers may include crosslinking or other linking agents that may result in polymers that do not form gels in solvents and may be soluble or partially soluble in some solvents.

Crosslinking agents are typically compounds having at least two functional groups that are selected from a halogen group, carbonyl group, epoxy group, ester group, acid anhydride group, acid halide group, isocyanate group, vinyl group, and chloroformate group. The crosslinking agent may be attached to the carbon backbone or to a nitrogen of the amine compound, amine monomer or residue thereof.

Examples of crosslinking agents that are suitable for synthesis of the polymers of the present invention include, but are not limited to, one or more multifunctional crosslinking agents such as: dihaloalkanes, haloalkyloxiranes, alkyloxirane sulfonates, di(haloalkyl)amines, tri(haloalkyl)amines, diepoxides, triepoxides, tetraepoxides, bis(halomethyl)benzenes, tri(halomethyl)benzenes, tetra(halomethyl)benzenes, epihalohydrin, epichlorohydrin, epibromohydrin poly(epichlorohydrin), (iodomethyl)oxirane, glycidyl tosylate, glycidyl 3-nitrobenzenesulfonate, 4-tosyloxy-1,2-epoxybutane, bromo-1,2-epoxybutane, 1,2-dibromoethane, 1,3-dichloropropane, 1,2-dichloroethane, 1-bromo-2-chloroethane, 1,3-dibromopropane, bis(2-chloroethyl)amine, tris(2-chloroethyl)amine, and bis(2-chloroethyl)methylamine, 1,3-butadiene diepoxide, 1,5-hexadiene diepoxide, diglycidyl ether, 1,2,7,8-diepoxyoctane, 1,2,9,10-diepoxydecane, ethylene glycol diglycidyl ether, propylene glycol diglycidyl ether, 1,4-butanediol diglycidyl ether, 1,2 ethanedioldiglycidyl ether, glycerol diglycidyl ether, 1,3-diglycidyl glyceryl ether, N,N-diglycidylaniline, neopentyl glycol diglycidyl ether, diethylene glycol diglycidyl ether, 1,4-bis(glycidyloxy)benzene, resorcinol diglycidyl ether, 1,6-hexanediol diglycidyl ether, trimethylolpropane diglycidyl ether, 1,4-cyclohexanedimethanol diglycidyl ether, 1,3-bis-(2,3-epoxypropyloxy)-2-(2,3-dihydroxypropyloxy)propane, 1,2-cyclohexanedicarboxylic acid diglycidyl ester, 2,2′-bis(glycidyloxy)diphenylmethane, bisphenol F diglycidyl ether, 1,4-bis(2′,3′-epoxypropyl)perfluoro-n-butane, 2,6-di(oxiran-2-ylmethyl)-1,2,3,5,6,7-hexahydropyrrolo[3,4-f]isoindol-1,3,5,7-tetraone, bisphenol A diglycidyl ether, ethyl 5-hydroxy-6,8-di(oxiran-2-ylmethyl)-4-oxo-4-h-chromene-2-carboxylate, bis[4-(2,3-epoxy-propylthio)phenyl]-sulfide, 1,3-bis(3-glycidoxypropyl)tetramethyldisiloxane, 9,9-bis[4-(glycidyloxy)phenyl]fluorine, triepoxyisocyanurate, glycerol triglycidyl ether, N,N-diglycidyl-4-glycidyloxyaniline, isocyanuric acid (S,S,S)-triglycidyl ester, isocyanuric acid (R,R,R)-triglycidyl ester, triglycidyl isocyanurate, trimethylolpropane triglycidyl ether, glycerol propoxylate triglycidyl ether, triphenylolmethane triglycidyl ether, 3,7,14-tris[[3-(epoxypropoxy)propyl]dimethylsilyloxy]-1,3,5,7,9,11,14-heptacyclopentyltricyclo[7.3.3.15,11]heptasiloxane, 4,4′-methylenebis(N,N-diglycidylaniline), bis(halomethyl)benzene, bis(halomethyl)biphenyl and bis(halomethyl)naphthalene, toluene diisocyanate, acrylol chloride, methyl acrylate, ethylene bisacrylamide, pyrometallic dianhydride, succinyl dichloride, dimethylsuccinate. When the crosslinking agent is an alkylhalide compound, a base can be used to scavenge the acid formed during the reaction. Inorganic or organic bases are suitable. NaOH is preferred. The base to crosslinking agent ratio is preferably between about 0.5 to about 2.

In some embodiments, the crosslinking agents may be introduced into the polymerization reaction in an amount of from 0.5 to 25 wt. %, such as from about 2 to about 15 wt. %, from about 2 to about 12 wt. %, from about 3 to about 10 wt. %, or from about 3 to about 6 wt. %, such as 2, 3, 4, 5, 6 wt %. The amount of crosslinking agent necessary may depend on the extent of branching within the amine compound.

In some embodiment the molecular weight of the amine polymers, may be typically at least about 1000. For example, the molecular weight may be from about 1000 to about 1,000,000, such as about 1000 to about 750,000, about 1000 to about 500,000, about 1000 to about 250,000, about 1000 to about 100,000 such as less than 750,000, less than 500,000, 250,000 or less than 100,000.

In some embodiments, the pharmaceutical composition of the present invention comprises an amine polymer comprising or derived from amine compounds represented by Formula VII where R′ independently represents a H radical or alkyl radical, q and r are 0 and p is 2, m independently represents an integer from 3-6, such as 3, 4, 5 or 6; and 2-6 wt. % crosslinking agent or residue thereof, such as 2 wt. %, 3 wt. %, 4 wt. %, 5 wt. % or 6 wt. % crosslinking agent, where the crosslinking agent is epichlorohydrin, poly(epichlorohydrin), 1,2-dibromoethane, tris(2-chloroethyl)amine or 1,4-butanediol diglycidyl ether. Another pharmaceutical composition embodiment of the present invention comprises an amine polymer comprising or derived from amine compounds represented by Formula VII where R′ independently represents a H radical or alkyl, radical, q is 0 and r and p both are 2, m independently represents an integer from 3-6, such as 3, 4, 5 or 6, and crosslinked with a crosslinking agent as defined above in this paragraph. A further pharmaceutical composition embodiment of the present invention comprises an amine polymer comprising or derived from amine compounds represented by Formula VII where R′ independently represents a H radical or alkyl, radical, q, r and p are each 2, m independently represents an integer from 3-6, such as 3, 4, 5 or 6, and crosslinked with a crosslinking agent as defined above in this paragraph.

A further pharmaceutical composition of the present invention comprises an amine polymer comprising or derived from amine compounds represented by Formula VII where R′ independently represents a H radical; p, q, and r independently represent either 0 or 2, m is 3 or 4, and 3-6 wt. % crosslinking agent or residue thereof, such as 3 wt. %, 4 wt. %, 5 wt. % or 6 wt. % crosslinking agent, where the crosslinking agent is epichlorohydrin, or 1,2-dibromoethane.

Another pharmaceutical composition of the present invention comprises an amine polymer comprising or derived from amine compounds represented by Formula IX where R′ independently represents a H radical or alkyl radical, q and r are 0 and p is 2, m independently represents an integer from 3-6, such as 3, 4, 5 or 6; and 2-6 wt. % crosslinking agent or residue thereof, such as 2 wt. %, 3 wt. %, 4 wt. %, 5 wt. % or 6 wt. % crosslinking agent, where the crosslinking agent is epichlorohydrin, poly(epichlorohydrin), 1,2-dibromoethane, tris(2-chloroethyl)amine or 1,4-butanediol diglycidyl ether. Another pharmaceutical composition embodiment of the present invention comprises an amine polymer comprising or derived from amine compounds represented by Formula IX where R′ independently represents a H radical or alkyl, radical, q is 0 and r and p both are 2, m independently represents an integer from 3-6, such as 3, 4, 5 or 6, and crosslinked with a crosslinking agent as defined above in this paragraph. A further pharmaceutical composition embodiment of the present invention comprises an amine polymer comprising or derived from amine compounds represented by Formula IX where R′ independently represents a H radical or alkyl, radical, q, r and p are each 2, m independently represents an integer from 3-6, such as 3, 4, 5 or 6, and crosslinked with a crosslinking agent as defined above in this paragraph. Preferred pharmaceutical composition of an embodiment of the present invention comprises an amine polymer comprising or derived from amine compounds represented by Formula IX where R′ represents a H radical; p, q, and r independently represent either 0 or 2, m is 3 or 4, and 3-6 wt. % crosslinking agent or residue thereof, such as 3 wt. %, 4 wt. %, 5 wt. % or 6 wt. % crosslinking agent, where the crosslinking agent is epichlorohydrin, or 1,2-dibromoethane.

Another pharmaceutical composition of the present invention comprises an amine polymer comprising or derived from amine compounds represented by Formula XI where R′ independently represents a H radical or alkyl radical, q and r are 0 and p is 2, m independently represents an integer from 3-6, such as 3, 4, 5 or 6; and 2-6 wt. % crosslinking agent or residue thereof, such as 2 wt. %, 3 wt. %, 4 wt. %, 5 wt. % or 6 wt. % crosslinking agent, where the crosslinking agent is epichlorohydrin, poly(epichlorohydrin), 1,2-dibromoethane, tris(2-chloroethyl)amine or 1,4-butanediol diglycidyl ether. Another pharmaceutical composition embodiment of the present invention comprises an amine polymer comprising or derived from amine compounds represented by Formula XI where R′ independently represents a H radical or alkyl, radical, q is 0 and r and p both are 2, m independently represents an integer from 3-6, such as 3, 4, 5 or 6, and crosslinked with a crosslinking agent as defined above in this paragraph. A further pharmaceutical composition embodiment of the present invention comprises an amine polymer comprising or derived from amine compounds represented by Formula XI where R′ independently represents a H radical or alkyl radical, q, r and p are each 2, m independently represents an integer from 3-6, such as 3, 4, 5 or 6, and crosslinked with a crosslinking agent as defined above in this paragraph. Preferred pharmaceutical composition of an embodiment of the present invention comprises an amine polymer comprising or derived from amine compounds represented by Formula XI where R′ represents a H radical; p, q, and r independently represent either 0 or 2, m is 3 or 4, and 3-6 wt. % crosslinking agent or residue thereof, such as 3 wt. %, 4 wt. %, 5 wt. % or 6 wt. % crosslinking agent, where the crosslinking agent is epichlorohydrin, or 1,2-dibromoethane.

The polymers of some embodiments may be formed using a polymerization initiator. Generally, any initiator may be used including cationic and radical initiators. Some examples of suitable initiators that may be used include: the free radical peroxy and azo type compounds, such as azodiisobutyronitrile, azodiisovaleronitrile, dimethylazodiisobutyrate, 2,2′-azobis(isobutyronitrile), 2,2′-azobis(N,N′-dimethyleneisobutyramidine)dihydrochloride, 2,2′-azobis(2-amidinopropane)dihydrochloride, 2,2′-azobis(N,N′-dimethyleneisobutyramidine), 1,1′-azobis(1-cyclohexanecarbo-nitrile), 4,4′-azobis(4-cyanopentanoic acid), 2,2′-azobis(isobutyramide) dihydrate, 2,2′-azobis(2-methylpropane), 2,2′-azobis(2-methylbutyronitrile), VAZO 67, cyanopentanoic acid, the peroxy pivalates, dodecylbenzene peroxide, benzoyl peroxide, di-t-butyl hydroperoxide, t-butyl peracetate, acetyl peroxide, dicumyl peroxide, cumyl hydroperoxide, dimethyl bis(butylperoxy) hexane.

In some embodiments, any of the nitrogen atoms within the amine compounds or residues thereof according to embodiments of the invention may optionally be quaternized to yield the corresponding positively charged tertiary nitrogen group, such as for example, an ammonium or substituted ammonium group. Any one or more of the nitrogen atoms in the amine compound or residue thereof may be quaternized and such quaternization, when present, is not limited to or required to include terminal amine nitrogen atoms. In some embodiments, this quaternization may result in additional network formation and may be the result of addition of crosslinking, linking or amine reactive groups to the nitrogen. The ammonium groups may be associated with a pharmaceutically acceptable counterion.

In some embodiments, compounds of the invention may be partially or fully quaternized, including protonated, with a pharmaceutically acceptable counterion, which may be organic ions, inorganic ions, or a combination thereof. Examples of some suitable inorganic ions include halides (e.g., chloride, bromide or iodide) carbonates, bicarbonates, sulfates, bisulfates, hydroxides, nitrates, persulfates and sulfites. Examples of some suitable organic ions include acetates, ascorbates, benzoates, citrates, dihydrogen citrates, hydrogen citrates, oxalates, succinates, tartrates, taurocholates, glycocholates, and cholates. Preferred ions include chlorides and carbonates.

In some embodiments, compounds and polymers of the invention may be protonated such that the fraction of protonated nitrogen atoms is from 1 to 25%, preferably 3 to 25%, more preferably 5 to 15%.

In one embodiment, the pharmaceutically acceptable amine compound is a polymer in protonated form and comprises a carbonate anion. In one embodiment the pharmaceutically acceptable amine compound is a polymer in protonated form and comprises a mixture of carbonate and bicarbonate anions.

In some embodiments, compounds of the invention are characterized by their ability to bind ions. Preferably the compounds of the invention bind anions, more preferably they bind phosphate and/or oxalate, and most preferably they bind phosphate ions. For illustration, anion-binding compounds and especially phosphate-binding compounds will be described; however, it is understood that this description applies equally, with appropriate modifications that will be apparent to those of skill in the art, to other ions and solutes. Compounds may bind an ion, e.g., an anion when they associate with the ion, generally though not necessarily in a noncovalent manner, with sufficient association strength, that at least a portion of the ion remains bound under the in vitro or in vivo conditions in which the polymer is used for sufficient time to effect a removal of the ion from solution or from the body. A target ion may be an ion to which the compound binds, and usually refers to the ion whose binding to the compound is thought to produce the therapeutic effect of the compound and may be an anion or a cation. A compound of the invention may have more than one target ion.

For example, some of the polymers described herein exhibit phosphate binding properties. Phosphate binding capacity is a measure of the amount of phosphate ion a phosphate binder can bind in a given solution.

For example, binding capacities of phosphate binders can be measured in vitro, e.g., in water or in saline solution, or in vivo, e.g., from phosphate urinary excretion, or ex vivo, for example using aspirate liquids, e.g., chyme obtained from lab animals, patients or volunteers. Measurements can be made in a solution containing only phosphate ion, or at least no other competing solutes that compete with phosphate ions for binding to the polymer resin. In these cases, a non interfering buffer may be used. Alternatively, measurements can be made in the presence of other competing solutes, e.g., other ions or metabolites, that compete with phosphate ions (the target solute) for binding to the resin.

Ion binding capacity for a compound can be measured as indicated in the Examples. Some embodiments have a phosphate binding capacity of which can be greater than about 0.2, 0.5, 1.0, 1.5, 2.0, 2.5, 3.0, 3.5, 4.0, 5.0, 6.0, 8.0, 10.0, 12, 14, 16, 18 or greater than about 20 mmol/g. In some embodiments, the in vitro phosphate binding capacity of compounds of the invention for a target ion is greater than about 0.5 mmol/g, preferably greater than about 2.5 mmol/g, even more preferably greater than about 3 mmol/g, even more preferably greater than about 4 mmol/g, and yet even more preferably greater than about 6 mmol/g. In some embodiments, the phosphate binding capacity can range from about 0.2 mmol/g to about 20 mmol/g, such as about 0.5 mmol/g to about 10 mmol/g, preferably from about 2.5 mmol/g to about 8 mmol/g, and even more preferably from about 3 mmol/g to about 6 mmol/g.

In some embodiments, compounds and compositions of the invention may reduce urinary phosphorous of a patient in need thereof by 5-100%, such as 10-75%, 25-65%, or 45-60%. Some embodiments may reduce urinary phosphorous by greater than 10%, greater than 20%, greater than 30%, greater than 40%, greater than 45%, greater than 50% or greater than 60%.

Several techniques are known in the art to determine the phosphate binding capacity. Examples of suitable techniques are described in the Examples section below.

When crosslinked, some embodiments of compounds of the invention form a gel in a solvent, such as in a simulated gastrointestinal medium or a physiologically acceptable medium.

### Core-Shell Compositions

One aspect of the invention is core-shell compositions comprising a polymeric core and shell. In some embodiments, the polymeric core comprises of the crosslinked polymers described herein. The shell material can be chemically anchored to the core material or physically coated. In the former case, the shell can be grown on the core component through chemical means, for example by: chemical grafting of shell polymer to the core using living polymerization from active sites anchored onto the core polymer; interfacial reaction, i.e., a chemical reaction located at the core particle surface, such as interfacial polycondensation; and using block copolymers as suspending agents during the core particle synthesis.

The interfacial reaction and use of block polymers are preferred techniques when chemical methods are used. In the interfacial reaction pathway, typically, the periphery of the core particle is chemically modified by reacting small molecules or macromolecules on the core interface. For example, an amine containing ion-binding core particle is reacted with a polymer containing amine reactive groups such as epoxy, isocyanate, activated esters, halide groups to form a crosslinked shell around the core.

In another embodiment, the shell is first prepared using interfacial polycondensation or solvent coacervation to produce capsules. The interior of the capsule is then filled up with core-forming precursors to build the core within the shell capsule.

In some embodiments, using the block copolymer approach, an amphiphilic block copolymer can be used as a suspending agent to form the core particle in an inverse or direct suspension particle forming process. When an inverse water-in-oil suspension process is used, then the block copolymer comprises a first block soluble in the continuous oil phase and another hydrophilic block contains functional groups that can react with the core polymer. When added to the aqueous phase, along with core-forming precursor, and the oil phase, the block copolymer locates to the water-in-oil interface and acts as a suspending agent. The hydrophilic block reacts with the core material, or co-reacts with the core-forming precursors. After the particles are isolated from the oil phase, the block copolymers form a thin shell covalently attached to the core surface. The chemical nature and length of the blocks can be varied to vary the permeation characteristics of the shell towards solutes of interest.

When the shell material is physically adsorbed on the core material, well known techniques of microencapsulation such as solvent coacervation, fluidized bed spray coater, or multiemulsion processes can be used. A preferred method of microencapsulation is the fluidized bed spray coater in the Wurster configuration. In yet another embodiment, the shell material is only acting temporarily by delaying the swelling of the core particle while in the mouth and esophagus, and optionally disintegrates in the stomach or duodenum. The shell is then selected in order to hinder the transport of water into the core particle, by creating a layer of high hydrophobicity and very low liquid water permeability.

In one embodiment the shell material carries negative charges while being in the milieu of use. Not being limited to one mechanism of action, it is thought that negatively charged shell material coated on anion-binding beads enhance the binding of small inorganic ions with a low charge density (such as phosphate) over competing ions with greater valency or size. Competing anions such as citrate, bile acids and fatty acids among others, may thus have a lesser relative affinity to the anion binding core possibly as a result of their limited permeability across the shell.

Preferred shell materials are polymers carrying negative charges in the pH range typically found in the intestine. Examples include, but are not limited to, polymers that have pendant acid groups such as carboxylic, sulfonic, hydrosulfonic, sulfamic, phosphoric, hydrophosphoric, phosphonic, hydrophosphonic, phosphoramidic, phenolic, boronic and a combination thereof. The polymer can be protonated or unprotonated; in the latter case the acidic anion can be neutralized with pharmaceutically acceptable cations such as Na, K, Li, Ca, Mg, and NH₄.

In another embodiment the polyanion can be administered as a precursor that ultimately activates as a polyanion: for instance certain labile ester or anhydride forms of either polysulfonic or polycarboxylic acids are prone to hydrolysis in the acidic environment of the stomach and can convert to the active anions.

The shell polymers can be either linear, branched, hyperbranched, segmented (i.e. backbone polymer arranged in sequence of contiguous blocks of which at least one contains pendant acidic groups), comb-shaped, star-shaped or crosslinked in a network, fully and semi-interpenetrated network (IPN). The shell polymers are either random or blocky in composition and either covalently or physically attached to the core material. Examples of such shell polymers include, but are not limited to acrylic acid homopolymers or copolymers, methacrylic acid homopolymers or copolymers, and copolymers of methacrylate and methacrylic acid. Examples of such polymers are copolymers of methylmethacrylate and methacrylic acid and copolymers of ethylacrylate and methacrylic acid, sold under the tradename Eudragit (Rohm GmbH & Co. KG): examples of which include Eudragit L100-55 and Eudragit L100 (a methylmethacrylate-methacrylic acid (1:1) copolymer, Degussa/Rohm), Eudragit L30-D55, Eudragit S 100-55 and Eudragit FS 30D, Eudragit S 100 (a methylmethacrylate-methacrylic acid (2:1) copolymer), Eudragit LD-55 (an ethylacrylate-methacrylic acid (1:1) copolymer), copolymers of acrylates and methacrylates with quaternary ammonium groups, sold under the tradenames Eudragit RL and Eudragit RS, and a neutral ester dispersion without any functional groups, sold under the tradename Eudragit NE30-D.

Additional shell polymers include: poly(styrene sulfonate), Polycarbophil®; Polyacrylic acid(s); carboxymethyl cellulose, cellulose acetate phthalate, hydroxypropyl methylcellulose phthalate as sold under the tradename HP-50 and HP-55 (Shin-Etsu Chemical Co., Ltd.), cellulose acetate trimellitate, cellulose acetate, cellulose acetate butyrate, cellulose acetate propionate, ethyl cellulose, cellulose derivatives, such as hydroxypropylmethylcellulose, methylcellulose, hydroxylethylcellulose, hydroxyethylmethylcellulose, hydroxylethylethylcellulose and hydroxypropylethylcellulose and cellulose derivatives such as cellulose ethers useful in film coating formulations, polyvinyl acetate phthalate, carrageenan, alginate, or poly(methacrylic acid) esters, acrylic/maleic acid copolymers, styrene/maleic acid polymers, itaconic acid/acrylic copolymers, and fumaric/acrylic acid copolymers, polyvinyl acetal diethylaminoacetate, as sold under the tradename AEA (Sankyo Co., Ltd.), methylvinylether/maleic acid copolymers and shellac.

In some preferred embodiments the shell polymers are selected amongst pharmaceutically acceptable polymers such as Eudragit L100-55 and Eudragit L100 (a methylmethacrylate-methacrylic acid (1:1) copolymer, Degussa/Rohm), Carbopol 934 (polyacrylic acid, Noveon), C-A-P NF (cellulose acetate phthalate—Eastman), Eastacryl (methacrylic acid esters-1-Eastman), Carrageenan and Alginate (FMC Biopolymer), Anycoat—P (Samsung Fine Chemicals—HPMC Phthalate), or Aqualon (carboxymethyl cellulose—Hercules), methylvinylether/maleic acid copolymers (Gantrez), and styrene/maleic acid (SMA).

The shell can be coated by a variety of methods. In one embodiment, the shell materials are added in the drug formulation step as an active excipient; for example, the shell material can be included in a solid formulation as a powder, which is physically blended with the phosphate-binding polymer and other excipients, optionally granulated, and compressed to form a tablet. Thus, in some embodiments, the shell material need not cover the core material in the drug product. For example, the acidic shell polymer may be added together with the anion binding core polymer formulated in the shape of a tablet, capsule, gel, liquid, etc, wafer, extrudates and the shell polymer can then dissolve and distribute itself uniformly as a shell coating around the core while the drug product equilibrates in the mouth, esophagus or ultimately in the site of action, i.e. the GI tract.

In some embodiments, the shell is a thin layer of shell polymer. The layer can be a molecular layer of polyanion on the core particle surface. The weight to core ratio can be between about 0.0001% to about 30%, preferably comprised between about 0.01% to about 5%, such as between about 0.1% to about 5%.

Preferably the shell polymers low minimum in molecular weight such that they do not freely permeate within the core pore volume nor elute from the core surface. Preferably the molecular weight of the shell acidic polymer Mw is about 1000 g/mole, more preferably about 5000 g/mole, and even more preferably about 20,000 g/mole

The anionic charge density of the shell material (as prevailing in the milieu of use) is may be between 0.5 mEq/gr to 22 mEq/gr, preferably 2 mEq/gr to 15 mEq/gr. If a coating process is used to form the shell on the polymer particles as part of the manufacture of the dosage form, then procedures known from those skilled-in-the-art in the pharmaceutical industry are applicable. In a preferred embodiment, the shell is formed in a fluidized bed coater (Wurster coater). In an alternate embodiment, the shell is formed through controlled precipitation or coacervation, wherein the polymer particles are suspended in a polymer solution, and the solvent properties are changed in such a way as to induce the polymer to precipitate onto or coat the polymer particles.

Suitable coating processes include the procedures typically used in the pharmaceutical industry. Typically, selection of the coating method is dictated by a number of parameters, that include, but are not limited to the form of the shell material (bulk, solution, emulsion, suspension, melt) as well as the shape and nature of the core material (spherical beads, irregular shaped, etc.), and the amount of shell deposited. In addition, the cores may be coated with one or more shells and may comprise multiple or alternating layers of shells.

### Treatment of Phosphate Imbalance Disorders and Renal Diseases

The term “phosphate imbalance disorder” as used herein refers to conditions in which the level of phosphorus present in the body is abnormal. One example of a phosphate imbalance disorder includes hyperphosphatemia. The term “hyperphosphatemia” as used herein refers to a condition in which the element phosphorus is present in the body at an elevated level. Typically, a patient is often diagnosed with hyperphosphatemia if the blood phosphate level is, for example, above about 4.0 or 4.5 milligrams per deciliter of blood, for example above about 5.0 mg/dl, such as above about 5.5 mg/dl, for example above 6.0 mg/dl, and/or glomerular filtration rate is reduced to, for example, more than about 20%. The present invention may also be used to treat patients suffering from hyperphosphatemia in End Stage Renal Disease and who are also receiving dialysis treatment (e.g., hemodialysis or peritoneal dialysis).

Other diseases that can be treated with the methods, compositions, and kits of the present invention include hypocalcemia, hyperparathyroidism, depressed renal synthesis of calcitriol, tetany due to hypocalcemia, renal insufficiency, and ectopic calcification in soft tissues including calcifications in joints, lungs, kidney, conjuctiva, and myocardial tissues. Also, the present invention can be used to treat Chronic Kidney Disease (CKD), End Stage Renal Disease (ESRD) and dialysis patients, including prophylactic treatment of any of the above.

Also, the polymers, compounds and compositions described herein can be used as an adjunct to other therapies e.g. those employing dietary control of phosphorus intake, dialysis inorganic metal salts and/or other polymer resins.

The compositions of the present invention are also useful in removing chloride, bicarbonate, oxalate, and bile acids from the gastrointestinal tract. Polymers removing oxalate ions find use in the treatment of oxalate imbalance disorders, such as such as oxalosis or hyperoxaluria that increases the risk of kidney stone formation. Polymers removing chloride ions find use in treating acidosis, heartburn, acid reflux disease, sour stomach or gastritis, for example. In some embodiments, the compositions of the present invention are useful for removing fatty acids, bilirubin, and related compounds. Some embodiments may also bind and remove high molecular weight molecules like proteins, nucleic acids, vitamins or cell debris.

The present invention provides methods, pharmaceutical compositions, and kits for the treatment of animal. The term “animal” or “animal subject” or “patient” as used herein includes humans as well as other mammals (e.g., in veterinary treatments, such as in the treatment of dogs or cats, or livestock animals such as pigs, goats, cows, horses, chickens and the like). One embodiment of the invention is a method of removing phosphate from the gastrointestinal tract of an animal by administering an effective amount of at least one of the crosslinked amine polymers described herein.

The term “treating” and its grammatical equivalents as used herein includes achieving a therapeutic benefit and/or a prophylactic benefit. By therapeutic benefit is meant eradication, amelioration, or prevention of the underlying disorder being treated. For example, in a hyperphosphatemia patient, therapeutic benefit includes eradication or amelioration of the underlying hyperphosphatemia. Also, a therapeutic benefit is achieved with the eradication, amelioration, or prevention of one or more of the physiological symptoms associated with the underlying disorder such that an improvement is observed in the patient, notwithstanding that the patient may still be afflicted with the underlying disorder. For example, administration of crosslinked amine polymers, described herein, to a patient suffering from renal insufficiency and/or hyperphosphatemia provides therapeutic benefit not only when the patient's serum phosphate level is decreased, but also when an improvement is observed in the patient with respect to other disorders that accompany renal failure and/or hyperphosphatemia like ectopic calcification and renal osteodistrophy. For prophylactic benefit, for example, the crosslinked amine polymers may be administered to a patient at risk of developing hyperphosphatemia or to a patient reporting one or more of the physiological symptoms of hyperphosphatemia, even though a diagnosis of hyperphosphatemia may not have been made.

The compositions may also be used to control serum phosphate in subjects with elevated phosphate levels, for example, by changing the serum level of phosphate towards a normal or near normal level, for example, towards a level that is within 10% of the normal level of a healthy patient.

Typically, the compounds can be administered before or after a meal, or with a meal. As used herein, “before” or “after” a meal is typically within two hours, preferably within one hour, more preferably within thirty minutes, most preferably within ten minutes of commencing or finishing a meal, respectively.

Other embodiments of the invention are directed towards pharmaceutical compositions comprising at least one of the compounds or a pharmaceutically acceptable salt of the compound, and one or more pharmaceutically acceptable excipients, diluents, or carriers and optionally additional therapeutic agents. The compounds may be lyophilized or dried under vacuum or oven before formulating.

The excipients or carriers are “acceptable” in the sense of being compatible with the other ingredients of the formulation and not deleterious to the recipient thereof. The formulations can conveniently be presented in unit dosage form and can be prepared by any suitable method. The methods typically include the step of bringing into association the agent with the excipients or carriers such as by uniformly and intimately bringing into association the polymer with the excipients or carriers and then, if necessary, dividing the product into unit dosages thereof.

The pharmaceutical compositions of the present invention include compositions wherein the crosslinked amine polymers are present in an effective amount, i.e., in an amount effective to achieve therapeutic and/or prophylactic benefit. The actual amount effective for a particular application will depend on the patient (e.g. age, weight) the condition being treated; and the route of administration.

The dosages of the amine compounds or polymers in animals will depend on the disease being, treated, the route of administration, and the physical characteristics of the animal being treated. Such dosage levels in some embodiments for either therapeutic and/or prophylactic uses may be from about 1 gm/day to about 30 gm/day, for example from about 2 gm/day to about 20 gm/day or from about 3 gm/day to about 7 gm/day. The dose of the compounds and polymers described herein can be less than about 50 gm/day, less than about 40 gm/day, less than about 30 gm/day, less than about 20 gm/day, and less than about 10 gm/day. Generally, it is preferred that the amine compounds or polymers are administered along with meals. The polymers may be administered one time a day, two times a day, or three times a day. Preferably the compounds are administered once a day with the largest meal.

Preferably, the amine compounds and polymers may be used for therapeutic and/or prophylactic benefits and can be administered alone or in the form of a pharmaceutical composition. The pharmaceutical compositions comprise the amine compounds and/or polymers, one or more pharmaceutically acceptable carriers, diluents or excipients, and optionally additional therapeutic agents. For example, the amine compounds and/or polymers of the present invention may be co-administered with other active pharmaceutical agents depending on the condition being treated. Examples of pharmaceutical agents that may be co-administered include, but are not limited to:

Other phosphate sequestrants suitable for use in the present invention include pharmaceutically acceptable lanthanum, calcium, aluminum, magnesium and zinc compounds, such as acetates, carbonates, oxides, hydroxides, citrates, alginates, and ketoacids thereof.

Calcium compounds, including calcium carbonate, acetate (such as PhosLo® calcium acetate tablets), citrate, alginate, and ketoacids, have been utilized for phosphate binding. The ingested calcium combines with phosphate to form insoluble calcium phosphate salts such as Ca₃(PO₄)₂, CaHPO₄, or Ca(H₂PO₄)₂.

Aluminium-based phosphate sequestrants, such as Amphojel® aluminium hydroxide gel, have also been used for treating hyperphosphatemia. These compounds complex with intestinal phosphate to form highly insoluble aluminium phosphate; the bound phosphate is unavailable for absorption by the patient.

The most commonly used lanthanide compound, lanthanum carbonate (Fosrenol®) behaves similarly to calcium carbonate.

Other phosphate sequestrants suitable for use in the present invention include pharmaceutically acceptable magnesium compounds. Various examples of pharmaceutically acceptable magnesium compounds are described in U.S. Provisional Application No. 60/734,593 filed Nov. 8, 2005, the entire teachings of which are incorporated herein by reference. Specific suitable examples include magnesium oxide, magnesium hydroxide, magnesium halides (e.g., magnesium fluoride, magnesium chloride, magnesium bromide and magnesium iodide), magnesium alkoxides (e.g., magnesium ethoxide and magnesium isopropoxide), magnesium carbonate, magnesium bicarbonate, magnesium formate, magnesium acetate, magnesium trisilicates, magnesium salts of organic acids, such as fumaric acid, maleic acid, acrylic acid, methacrylic acid, itaconic acid and styrenesulfonic acid, and a combination thereof.

Various examples of pharmaceutically acceptable zinc compounds are described in PCT Application No. PCT/US2005/047582 filed Dec. 29, 2005, the entire teachings of which are incorporated herein by references. Specific suitable examples of pharmaceutically acceptable zinc compounds include zinc acetate; zinc bromide, zinc caprylate, zinc carbonate, zinc chloride, zinc citrate, zinc formate, zinc hexafluorosilicate, zinc iodate, zinc iodide, zinc iodide-starch, zinc lactate, zinc nitrate, zinc oleate, zinc oxalate, zinc oxide, calamine (zinc oxide with a small proportion of ferric oxide), zinc p-phenolsulfonate, zinc propionate, zinc salicylate, zinc silicate, zinc stearate, zinc sulfate, zinc sulfide, zinc tannate, zinc tartrate, zinc valerate and zinc ethylenebis(dithiocarbamate). Another example includes poly(zinc acrylate).

When referring to any of the above-mentioned phosphate sequestrants, it is to be understood that mixtures, polymorphs and solvates thereof are encompassed.

In some embodiments, a mixture of the phosphate sequestrants described above can be used in the invention in combination with pharmaceutically acceptable ferrous iron salts:

In other embodiments, the phosphate sequestrant used in combination with compounds of the present invention is not a pharmaceutically acceptable magnesium compound. In yet other embodiments, the phosphate sequestrant used in combination with the pharmaceutically acceptable amine compounds and/or polymers is not a pharmaceutically acceptable zinc compound.

The invention also includes methods and pharmaceutical compositions directed to a combination therapy of the amine compounds and/or polymers in combination with a phosphate transport inhibitor; an HMG-CoA reductase inhibitor, such as a statin; or an alkaline phosphatase inhibitor. Alternatively, a mixture of the amine compounds and/or polymers is employed together with a phosphate transport inhibitor; an HMG-CoA reductase inhibitor, such as a statin; or an alkaline phosphatase inhibitor.

Suitable examples of phosphate transport inhibitors can be found in co-pending U.S. Application Publication Nos. 2004/0019113 and 2004/0019020 and WO 2004/085448, the entire teachings of each of which are incorporated herein by reference.

Suitable examples of HMG-CoA reductase inhibitors for the combination therapy of the invention include lovastatin (mevinolin) (e.g., Altocor® and Mevacor®) and related compounds; pravastatin (e.g., Pravachol®, Selektine®, and Lipostat®) and related compounds; simvastatin (e.g., Zocor®) and related compounds. Other HMG-CoA reductase inhibitors which can be employed in the present invention include fluvastatin (e.g., Lescol®); cerivastatin (e.g., Baycol® and Lipobay®); atorvastatin (e.g., Zarator® and Lipitor®); pitavastatin; rosuvastatin (visastatin) (e.g., Crestor®); quinoline analogs of mevalonolactone and derivatives thereof (see U.S. Pat. No. 5,753,675, the entire teachings of which are incorporated herein by reference); pyrazole analogs of mevalonolactone derivatives (see U.S. Pat. No. 4,613,610, the entire teachings of which are incorporated herein by reference); indene analogs of mevalonolactone derivatives (see WO 86/03488, the entire teachings of which are incorporated herein by reference); 6-[2-(substituted-pyrrol-1-yl)-alkyl)pyran-2-ones and derivatives thereof (see U.S. Pat. No. 4,647,576, the entire teachings of which are incorporated herein by reference); imidazole analogs of mevalonolactone (see WO 86/07054, the entire teachings of which are incorporated herein by reference); 3-hydroxy-4(dihydroxooxophosphorio)butanoic acid derivatives (see French Patent No. 2,596,393, the entire teachings of which are incorporated herein by reference); naphthyl analogs of mevalonolactone (see U.S. Pat. No. 4,686,237, the entire teachings of which are incorporated herein by reference); octahydronaphthalenes (see U.S. Pat. No. 4,499,289, the entire teachings of which are incorporated herein by reference); and quinoline and pyridine derivatives (see U.S. Pat. Nos. 5,506,219 and 5,691,322, the entire teachings of which are incorporated herein by reference). A statin, such as atorvastatin, fluvastatin, lovastatin, pravastatin, simvastatin, rosuvastatin, cerivastatin and pitavastatin, is preferred.

A large variety of organic and inorganic molecules are inhibitors to alkaline phosphatase (ALP) (see, for example, U.S. Pat. No. 5,948,630, the entire teachings of which are incorporated herein by reference). Examples of alkaline phosphatase inhibitors include orthophosphate, arsenate, L-phenylalanine, L-homoarginine, tetramisole, levamisole, L-p-Bromotetramisole, 5,6-Dihydro-6-(2-naphthyl) imidazo-[2,1-b]thiazole (napthyl) and derivatives thereof. The preferred inhibitors include, but are not limited to, levamisole, bromotetramisole, and 5,6-Dihydro-6-(2-naphthyl)imidazo-[2,1-b]thiazole and derivatives thereof.

This co-administration can include simultaneous administration of the two agents in the same dosage form, simultaneous administration in separate dosage forms, and separate administration. For example, for the treatment of hyperphosphatemia, the crosslinked amine polymers may be co-administered with calcium salts which are used to treat hypocalcemia resulting from hyperphosphatemia.

The pharmaceutical compositions of the invention can be formulated as a tablet, sachet, slurry, food formulation, troche, capsule, elixir, suspension, syrup, wafer, chewing gum or lozenge.

Preferably, the amine compounds or polymers or the pharmaceutical compositions comprising the amine compounds or polymers is administered orally. Illustrative of suitable methods, vehicles, excipients and carriers are those described, for example, in Remington's Pharmaceutical Sciences, 18th ed. (1990), the contents of which is incorporated herein by reference.

Pharmaceutical compositions for use in accordance with the present invention may be formulated in conventional manner using one or more physiologically acceptable carriers comprising excipients and auxiliaries which facilitate processing of the active compounds into preparations which can be used pharmaceutically. Proper formulation is dependent upon the route of administration chosen. Suitable techniques for preparing pharmaceutical compositions of the amines are well known in the art.

In some embodiments the polymers of the invention are provided as pharmaceutical compositions in the form of chewable tablets. In addition to the active ingredient, the following types of excipients are commonly used: a sweetening agent to provide the necessary palatability, plus a binder where the former is inadequate in providing sufficient tablet hardness; a lubricant to minimize frictional effects at the die wall and facilitate tablet ejection; and, in some formulations a small amount of a disintegrant is added to facilitate mastication. In general excipient levels in currently-available chewable tablets are on the order of 3-5 fold of active ingredient(s) whereas sweetening agents make up the bulk of the inactive ingredients.

In some aspects of the invention, the polymer(s) provide mechanical and thermal properties that are usually performed by excipients, thus decreasing the amount of such excipients required for the formulation. In some embodiments the polymer or composition constitutes over about 30 wt. %, for example over about 40 wt. %, over about 50 wt. %, preferably over about 60 wt. %, over about 70 wt. %, more preferably over about 80 wt. %, over about 85 wt. % or over about 90 wt. % of the composition, the remainder comprising suitable excipient(s).

In some embodiments, the compressibility of the tablets is strongly dependent upon the degree of hydration (moisture content) of the polymer or compound. Preferably, the polymer or compound has a moisture content of about 5% by weight or greater, more preferably, the moisture content is from about 5% to about 9% by weight, and most preferably about 7% by weight. It is to be understood that in embodiments in which the polymer is hydrated, the water of hydration is considered to be a component of the polymer.

The tablet can further comprise one or more excipients, such as hardeners, glidants and lubricants, which are well known in the art. Suitable excipients include colloidal silicon dioxide, stearic acid, magnesium silicate, calcium silicate, sucrose, calcium stearate, glyceryl behenate, magnesium stearate, talc, zinc stearate and sodium stearylfumarate.

The tablet core of embodiments of the invention may be prepared by a method comprising the steps of: (1) hydrating or drying the aliphatic amine polymer to the desired moisture level; (2) blending the polymer with any excipients; and (3) compressing the blend using conventional tableting technology.

In some embodiments, the invention relates to a stable, swallowable coated tablet, particularly a tablet comprising a hydrophilic core, such as a tablet comprising the polymer, as described above. In one embodiment, the coating composition comprises a cellulose derivative and a plasticizing agent. The cellulose derivative is, preferably, hydroxypropylmethylcellulose (HPMC). The cellulose derivative can be present as an aqueous solution. Suitable hydroxypropylmethylcellulose solutions include those containing HPMC low viscosity and/or HPMC high viscosity. Additional suitable cellulose derivatives include cellulose ethers useful in film coating formulations. The plasticizing agent can be, for example, an acetylated monoglyceride such as diacetylated monoglyceride. The coating composition can further include a pigment selected to provide a tablet coating of the desired color. For example, to produce a white coating, a white pigment can be selected, such as titanium dioxide.

In one embodiment, the coated tablet of the invention can be prepared by a method comprising the step of contacting a tablet core of the invention, as described above, with a coating solution comprising a solvent, at least one coating agent dissolved or suspended in the solvent and, optionally, one or more plasticizing agents. Preferably, the solvent is an aqueous solvent, such as water or an aqueous buffer, or a mixed aqueous/organic solvent. Preferred coating agents include cellulose derivatives, such as hydroxypropylmethylcellulose. Typically, the tablet core is contacted with the coating solution until the weight of the tablet core has increased by an amount ranging from about 4% to about 6%, indicating the deposition of a suitable coating on the tablet core to form a coated tablet.

Other pharmaceutical excipients useful in the some compositions of the invention include a binder, such as microcrystalline cellulose, carbopol, providone and xanthan gum; a flavoring agent, such as mannitol, xylitol, maltodextrin, fructose, or sorbitol; a lubricant, such as vegetable based fatty acids; and, optionally, a disintegrant, such as croscarmellose sodium, gellan gum, low-substituted hydroxypropyl ether of cellulose, sodium starch glycolate. Such additives and other suitable ingredients are well-known in the art; see, e.g., Gennaro A R (ed), Remington's Pharmaceutical Sciences, 20th Edition.

In some embodiments the invention provides a pharmaceutical composition formulated as a chewable tablet, comprising a polymer described herein and a suitable excipient. In some embodiments the invention provides a pharmaceutical composition formulated as a chewable tablet, comprising a polymer described herein, a filler, and a lubricant. In some embodiments the invention provides a pharmaceutical composition formulated as a chewable tablet, comprising a polymer described herein, a filler, and a lubricant, wherein the filler is chosen from the group consisting of sucrose, mannitol, xylitol, maltodextrin, fructose, and sorbitol, and wherein the lubricant is a magnesium fatty acid salt, such as magnesium stearate.

In one embodiment, the polymer is pre-formulated with a high Tg/high melting point low molecular weight excipient such as mannitol, sorbose, sucrose in order to form a solid solution wherein the polymer and the excipient are intimately mixed. Methods of mixing such as extrusion, spray-drying, chill drying, lyophilization, or wet granulation are useful. Indication of the level of mixing is given by known physical methods such as differential scanning calorimetry or dynamic mechanical analysis.

In some embodiments the polymers of the invention are provided as pharmaceutical compositions in the form of liquid formulations. In some embodiments the pharmaceutical composition contains polymer dispersed in a suitable liquid excipient. Suitable liquid excipients are known in the art; see, e.g., Remington's Pharmaceutical Sciences.

In some embodiments, the pharmaceutical compositions may be in the form of a powder formulation packaged as a sachet that may be mixed with water or other ingestible liquid and administered orally as a drink (solution or suspension). In order to ensure that such formulations provide acceptable properties to the patient such as mouth feel and taste, a pharmaceutically acceptable anionic stabilizer may be included in the formulation.

Examples of suitable anionic stabilizers include anionic polymers such as: an anionic polypeptide, an anionic polysaccharide, or a polymer of one or more anionic monomers such as polymers of mannuronic acid, guluronic acid, acrylic acid, methacrylic acid, glucuronic acid glutamic acid or a combination thereof, and pharmaceutically acceptable salts thereof. Other examples of anionic polymers include cellulose, such as carboxyalkyl cellulose or a pharmaceutically acceptable salt thereof. The anionic polymer may be a homopolymer or copolymer of two or more of the anionic monomers described above. Alternatively, the anionic copolymer may include one or more anionic monomers and one or more neutral comonomers such as olefinic anionic monomers such as vinyl alcohol, acrylamide, and vinyl formamide.

Examples of anionic polymers include alginates (e.g. sodium alginate, potassium alginate, calcium alginate, magnesium alginate, ammonium alginate, and esters of alginate), carboxymethyl cellulose, polylactic acid, polyglutamic acid, pectin, xanthan, carrageenan, furcellaran, gum Arabic, karaya gum, gum ghatti, gum carob, and gum tragacanth. Preferred anionic polymers are alginates and are preferably esterified alginates such as a C2-C5-diol ester of alginate or a C3-C5 triol ester of alginate. As used herein an “esterified alginate” means an alginic acid in which one or more of the carboxyl groups have of the alginic acid are esterified. The remainder of the carboxylic acid groups in the alginate are optionally neutralized (partially or completely) as pharmaceutically acceptable salts. For example, propylene glycol alginate is an ester of alginic acid in which some of the carboxyl groups are esterified with propylene glycol, and the remainder of the carboxylic acid groups are optionally neutralized with pharmaceutically acceptable salts. More preferably, the anionic polymer is ethylene glycol alginate, propylene glycol alginate or glycerol alginate, with propylene glycol alginate even more preferred.

All publications and patent applications mentioned in this specification are herein incorporated by reference to the same extent as if each individual publication or patent application was specifically and individually indicated to be incorporated by reference.

It will be apparent to one of ordinary skill in the art that many changes and modification can be made to the disclosures presented herein without departing from the spirit or scope of the appended claims.

### EXAMPLES

As used herein, the following terms have the meanings ascribed to them unless specified otherwise.

DAB-4—1,4-Bis[bis(3-aminopropyl)amino]butane, commercially available from Aldrich.

DAB-8—1,4-Bis(bis(3-(bis(3-aminopropyl)amino)propyl)amino)butane, commercially available from SyMO-Chem.

DAB-16—1,4-Bis[bis[3-[bis[3-[bis(3-aminopropyl)amino]propyl]amino]propyl]amino]butane, commercially available from SyMO-Chem.

DAB-32—1,4-Bis[bis[3-[bis[3-[(bis[3-[bis(3-aminopropyl)amino]propyl]amino]propyl]amino]propyl]amino]butane, commercially available from SyMO-Chem.

DAB-64—1,4-Bis[bis[3-[bis[3-[bis[3-[bis[3-[bis(3-aminopropyl)amino]propyl]amino]propyl]amino]propyl]amino]propyl]amino]butane, commercially available from SyMO-Chem.

PAMAM—amidoethylethanolamine dendrimer with a 1,4 diaminobutane core 20% solution in methanol, commercially available from Dendritic NanoTechnologies, Inc. as DNT-103.

DAP-Am-4—N,N,N′,N′-Tetrakis(3-aminopropyl)-1,3-propanediamine, commercially available PolyOrg, Inc.

EPI—epichlorohydrin, commercially available from Aldrich.

Poly(epichlorohydrin)—commercially available from Aldrich.

TCA—tris(2-chloroethyl)amine, commercially available from Aldrich.

DBE—1,2-dibromoethane, commercially available from Aldrich.

BDDE—1,4-butanedioldiglycidyl ether, commercially available from Aldrich.

In Vitro Phosphate Binding—refers to the methods set forth below

In-Process Swelling Ratio—refers to the methods set forth below

### Examples 1-47

The amine polymers of examples 1-47 were prepared by stirring a solution of Amine and Solvent at room temperature, optionally, under nitrogen atmosphere, and adding a Crosslinker to form a gel. After curing and cooling to room temperature, the gel was broken into small pieces and suspended in water or methanol, stirred, and filtered. The filtered gel was resuspended in deionized water, stirred, and filtered. Optionally, the pH of the solution was adjusted appropriately with concentrated HCl. The solution was then filtered. The washed polymer was dried in a forced-air oven at 60 degrees C. to afford a dry weight of polymer.

Tables 1-10 provide the specific components and amounts for Examples 1-47. Also, in vitro phosphate binding data and swelling ratios for some of the examples are provided within Tables 1-10. Tables 11-28 provide data for the in vivo reduction of urinary phosphate.

TABLE 1

| INGREDIENT | | Ex. 1 | | Ex. 2 | | Ex. 3 | | Ex. 4 | Ex. 5 |
|---|---|---|---|---|---|---|---|---|---|
| Amine | DAB-4 | 47.48 | g | 47.48 | g | 49.46 | ml | — | — |

| | DAB-8 | — | | — | | — | — | | 10 | g |
|---|---|---|---|---|---|---|---|---|---|---|
| | DAB-16 | — | | — | | — | 10 | g | — | |
| | DAB-32 | — | | — | | — | — | | — | |
| | DAB-64 | — | | — | | — | — | | — | |
| | PAMAM | — | | — | | — | — | | — | |
| Solvent | Water | 50 | g | 25 | g | — | 10 | g | 10 | g |

| | | | | | | |
|---|---|---|---|---|---|---|
| Methanol | — | — | 25 | ml | — | — |

| | | | | | | | |
|---|---|---|---|---|---|---|---|
| Crosslinker | EPI | 11.73 | ml | — | — | — | — |

| | | | | | | |
|---|---|---|---|---|---|---|
| TCA | — | 36.15 | g | — | — | — |

| | | | | | | |
|---|---|---|---|---|---|---|
| DBE | — | — | 12.93 | g | — | — |

| | | | | | | | |
|---|---|---|---|---|---|---|---|
| BDDE | — | — | — | 2.12 | g | 2.12 | g |

| | | | | | | |
|---|---|---|---|---|---|---|
| In Vitro | 1 hour | — | — | — | — | — |
| Phosphate | 5 hour | — | — | — | — | — |
| Binding | | | | | | |
| (mmol/g) | | | | | | |

| | | | | | |
|---|---|---|---|---|---|
| In-Process Swelling | 9.83 | — | 13.74 | 5.7 | 20 |
| Ratio (mL/g) | | | | | |

TABLE 2

| INGREDIENT | | Ex. 6 | | Ex. 7 | | Ex. 8 | | Ex. 9 | | Ex. 10 | |
|---|---|---|---|---|---|---|---|---|---|---|---|
| Amine | DAB-4 | 25 | g | 75 | g | 20 | g | 20 | g | 20 | g |
| | DAB-8 | — | | — | | — | | — | | — | |
| | DAB-16 | — | | — | | — | | — | | — | |
| | DAB-32 | — | | — | | — | | — | | — | |
| | DAB-64 | — | | — | | — | | — | | — | |
| | PAMAM | — | | — | | — | | — | | — | |
| Solvent | Water | 16.4 | g | 75 | g | 80 | g | 80 | g | 80 | g |
| | Methanol | — | | — | | — | | — | | — | |
| Crosslinker | EPI | 18.32 | g | 18.54 | ml | 4.94 | ml | 9.89 | ml | 14.83 | ml |

| | | | | | | |
|---|---|---|---|---|---|---|
| | TCA | — | — | — | — | — |
| | DBE | — | — | — | — | — |
| | BDDE | — | — | — | — | — |
| In Vitro | 1 hour | — | — | — | — | — |
| Phosphate | 5 hour | — | — | — | — | — |
| Binding | | | | | | |
| (mmol/g) | | | | | | |

| | | | | | |
|---|---|---|---|---|---|
| In-Process Swelling | 6.08 | 7.37 | 15.05 | 3.68 | 2.33 |
| Ratio (mL/g) | | | | | |

TABLE 3

| INGREDIENT | | Ex. 11 | | Ex. 12 | | Ex. 13 | | Ex. 14 | | Ex. 15 | |
|---|---|---|---|---|---|---|---|---|---|---|---|
| Amine | DAB-4 | 20 | g | — | | — | | — | | — | |
| | DAB-8 | — | | 10 | g | — | | — | | — | |
| | DAB-16 | — | | — | | 10 | g | 30 | g | — | |
| | DAB-32 | — | | — | | — | | — | | 9.4 | g |
| | DAB-64 | — | | — | | — | | — | | — | |
| | PAMAM | — | | — | | — | | — | | — | |
| Solvent | Water | 80 | g | 10 | g | 10 | g | 30 | g | 10 | g |
| | Methanol | — | | — | | — | | — | | — | |
| Crosslinker | EPI | 19.76 | ml | 0.821 | ml | 0.821 | ml | 2.46 | ml | 0.386 | ml |

| | TCA | — | — | — | — | — |
|---|---|---|---|---|---|---|
| | DBE | — | — | — | — | — |
| | BDDE | — | — | — | — | — |
| In Vitro | 1 hour | — | — | — | 0.53 | — |
| Phosphate | 5 hour | — | — | — | 0.28 | — |
| Binding | | | | | | |
| (mmol/g) | | | | | | |

| | | | | | |
|---|---|---|---|---|---|
| In-Process Swelling | 1.71 | 4.6 | 4.6 | 4.7 | 6.4 |
| Ratio (mL/g) | | | | | |

TABLE 4

| INGREDIENT | | Ex. 16 | Ex. 17 | | Ex. 18 | | Ex. 19 | | Ex. 20 | |
|---|---|---|---|---|---|---|---|---|---|---|
| Amine | DAB-4 | — | — | | — | | — | | — | |
| | DAB-8 | — | 10.67 | g | 10 | g | — | | 10 | g |
| | DAB-16 | 10 g | — | | — | | 10 | g | — | |
| | DAB-32 | — | — | | — | | — | | — | |
| | DAB-64 | — | — | | — | | — | | — | |
| | PAMAM | — | — | | — | | — | | — | |
| Solvent | Water | 10 g | 10.67 | g | 10 | g | 10 | g | 10 | g |
| | Methanol | — | — | | — | | — | | — | |
| Crosslinker | EPI | — | 1.75 | ml | 3.29 | ml | 3.29 | ml | 1.23 | ml |

| | TCA | — | — | — | — | — |
|---|---|---|---|---|---|---|
| | DBE | — | — | — | — | — |
| | BDDE | — | — | — | — | — |
| In Vitro | 1 hour | — | — | 1.96 | 2.22 | 0.00 |
| Phosphate | 5 hour | — | — | 1.33 | 1.84 | 0.00 |
| Binding | | | | | | |
| (mmol/g) | | | | | | |

| | | | | | |
|---|---|---|---|---|---|
| In-Process Swelling | 2.23 | 3.9 | 2 | 1.6 | 6.4 |
| Ratio (mL/g) | | | | | |

TABLE 5

| INGREDIENT | | Ex. 21 | | Ex. 22 | | Ex. 23 | | Ex. 24 | | Ex. 25 | |
|---|---|---|---|---|---|---|---|---|---|---|---|
| Amine | DAB-4 | — | | — | | — | | — | | — | |
| | DAB-8 | — | | 10 | g | — | | 25 | g | — | |
| | DAB-16 | 10 | g | — | | 10 | g | — | | 25 | g |
| | DAB-32 | — | | — | | — | | — | | — | |
| | DAB-64 | — | | — | | — | | — | | — | |
| | PAMAM | — | | — | | — | | — | | — | |
| Solvent | Water | 10 | g | 10 | g | 10 | g | 25 | g | 25 | g |
| | Methanol | — | | — | | — | | — | | — | |
| Crosslinker | EPI | 1.23 | ml | 2.46 | ml | 2.46 | ml | 4.11 | ml | 4.11 | ml |

| | TCA | — | — | — | — | — |
|---|---|---|---|---|---|---|
| | DBE | — | — | — | — | — |
| | BDDE | — | — | — | — | — |
| In Vitro | 1 hour | 0.73 | 0.96 | 1.97 | 0.56 | 0.89 |
| Phosphate | 5 hour | 0.41 | 0.88 | 1.85 | 0.33 | 0.81 |
| Binding | | | | | | |
| (mmol/g) | | | | | | |

| | | | | | |
|---|---|---|---|---|---|
| In-Process Swelling | 2.8 | 2.6 | 1.9 | 4.4 | 2.75 |
| Ratio (mL/g) | | | | | |

TABLE 6

| INGREDIENT | | Ex. 26 | | Ex. 27 | | Ex. 28 | | Ex. 29 | | Ex. 30 | |
|---|---|---|---|---|---|---|---|---|---|---|---|
| Amine | DAB-4 | — | | 25 | g | 25 | g | 25 | g | — | |
| | DAB-8 | — | | — | | — | | — | | — | |
| | DAB-16 | 20 | g | — | | — | | — | | 30.1 | g |
| | DAB-32 | — | | — | | — | | — | | — | |
| | DAB-64 | — | | — | | — | | — | | — | |
| | PAMAM | — | | — | | — | | — | | — | |
| Solvent | Water | 20 | g | 25 | g | 25 | g | 25 | g | 30.1 | g |
| | Methanol | — | | — | | — | | — | | — | |
| Crosslinker | EPI | 1.64 | ml | 4.11 | ml | 8.22 | ml | 6.16 | ml | 2.47 | ml |

| | TCA | — | — | — | — | — |
|---|---|---|---|---|---|---|
| | DBE | — | — | — | — | — |
| | BDDE | — | — | — | — | — |
| In Vitro | 1 hour | 0.23 | — | 0.44 | — | 0.58 |
| Phosphate | 5 hour | 0.02 | — | 0.11 | — | 0.22 |
| Binding | | | | | | |
| (mmol/g) | | | | | | |

| | | | | | |
|---|---|---|---|---|---|
| In-Process Swelling | 4.77 | — | 5.2 | 17.2 | 5.9 |
| Ratio (mL/g) | | | | | |

TABLE 7

| INGREDIENT | | Ex. 31 | | Ex. 32 | Ex. 33 | | Ex. 34 | | Ex. 35 |
|---|---|---|---|---|---|---|---|---|---|
| Amine | DAB-4 | — | | — | — | | — | | — |
| | DAB-8 | — | | 7.86 g | 10 | g | — | | 10 g |
| | DAB-16 | 60 | g | — | — | | 10 | g | — |
| | DAB-32 | — | | — | — | | — | | — |
| | DAB-64 | — | | — | — | | — | | — |
| | PAMAM | — | | — | — | | — | | — |
| Solvent | Water | 60 | g | 7.86 g | 10 | g | 10 | g | 10 g |
| | Methanol | — | | — | — | | — | | — |
| Crosslinker | EPI | 4.93 | ml | — | 4.11 | ml | 4.11 | ml | — |

| | TCA | — | — | — | — | — |
|---|---|---|---|---|---|---|
| | DBE | — | — | — | — | — |
| | BDDE | — | — | — | — | — |
| In Vitro | 1 hour | 0.41 | 0.18 | 2.00 | 1.99 | 1.51 |
| Phosphate | 5 hour | 0.29 | 0.02 | 2.08 | 1.85 | 1.65 |
| Binding | | | | | | |
| (mmol/g) | | | | | | |

| | | | | | |
|---|---|---|---|---|---|
| In-Process Swelling | 5.3 | 3.85 | 1.3 | 1.22 | 1.05 |
| Ratio (mL/g) | | | | | |

TABLE 8

| INGREDIENT | | Ex. 36 | | Ex. 37 | | Ex. 38 | | Ex. 39 | | Ex. 40 | |
|---|---|---|---|---|---|---|---|---|---|---|---|
| Amine | DAB-4 | — | | — | | — | | — | | — | |
| | DAB-8 | — | | 10 | g | 10 | g | — | | — | |
| | DAB-16 | 10 | g | — | | — | | 10 | g | 10 | g |
| | DAB-32 | — | | — | | — | | — | | — | |
| | DAB-64 | — | | — | | — | | — | | — | |
| | PAMAM | — | | — | | — | | — | | — | |
| Solvent | Water | 10 | g | 10 | g | 10 | g | 10 | g | 10 | g |
| | Methanol | — | | — | | — | | — | | — | |
| Crosslinker | EPI | 5.48 | ml | 0.410 | ml | 0.205 | ml | 0.205 | ml | 0.410 | ml |

| | | | | | | |
|---|---|---|---|---|---|---|
| | TCA | — | — | — | — | — |
| | DBE | — | — | — | — | — |
| | BDDE | — | — | — | — | — |
| In Vitro | 1 hour | 1.34 | — | — | — | — |
| Phosphate | 5 hour | 1.32 | — | — | — | — |
| Binding | | | | | | |
| (mmol/g) | | | | | | |

| | | | | | |
|---|---|---|---|---|---|
| In-Process Swelling | 0.95 | — | — | — | — |
| Ratio (mL/g) | | | | | |

TABLE 9

| INGREDIENT | | Ex. 41 | | Ex. 42 | | Ex. 43 | | Ex. 44 | | Ex. 45 | |
|---|---|---|---|---|---|---|---|---|---|---|---|
| Amine | DAB-4 | — | | — | | — | | — | | — | |
| | DAB-8 | — | | 30 | g | — | | — | | 10.67 | g |
| | DAB-16 | — | | — | | 30 | g | 10 | g | — | |
| | DAB-32 | — | | — | | — | | — | | — | |
| | DAB-64 | 9 | g | — | | — | | — | | — | |
| | PAMAM | — | | — | | — | | — | | — | |
| Solvent | Water | 18 | g | 30 | g | 30 | g | 10 | g | 10.67 | g |
| | Methanol | — | | — | | — | | — | | — | |
| Crosslinker | EPI | 0.369 | ml | 2.46 | ml | 2.46 | ml | 1.64 | ml | 1.75 | ml |

| | | | | | | |
|---|---|---|---|---|---|---|
| | TCA | — | — | — | — | — |
| | DBE | — | — | — | — | — |
| | BDDE | — | — | — | — | — |
| In Vitro | 1 hour | 0.57 | — | — | — | — |
| Phosphate | 5 hour | 0.37 | — | — | — | — |
| Binding | | | | | | |
| (mmol/g) | | | | | | |

| | | | | | |
|---|---|---|---|---|---|
| In-Process Swelling | — | — | — | — | — |
| Ratio (mL/g) | | | | | |

TABLE 10

| INGREDIENT | | Ex. 46 | Ex. 47 |
|---|---|---|---|
| Amine | DAB-4 | — | — |
| | DAB-8 | — | — |
| | DAB-16 | — | — |
| | DAB-32 | — | — |
| | DAB-64 | — | — |
| | PAMAM | 0.550 g | 6 g of |
| | | | 20% |
| | | | solution in |
| | | | methanol |
| Solvent | Water | 1.1 g | 7 g |
| | Methanol | — | — |
| Crosslinker | EPI | 0.021 ml | 0.153 ml |
| | TCA | — | — |
| | DBE | — | — |
| | BDDE | — | — |
| In Vitro | 1 hour | — | — |
| Phosphate | 5 hour | — | — |
| Binding | | | |
| (mmol/g) | | | |

| | | |
|---|---|---|
| In-Process Swelling | — | — |
| Ratio (mL/g) | | |

### Examples 48-82

### Ex. 48

To a solution of DAB-16 (10.33 g) in deionized water (40 mL) Was added concentrated HCl (9.5 mL) until a solution pH 8.1. The solution was lyophilized to afford 11.9 g.

### Ex. 49

A solution of DAB-8 (7.18 g), formic acid (35 g of an 88% aqueous solution), and formaldehyde (18.11 g of a 37 wt % aqueous solution) was heated at 80 degrees C. for 24 h. After cooling to room temperature 50% aqueous NaOH was added to the reaction mixture until pH 13.5, followed by deionized water (30 mL). The reaction mixture was extracted with methylene chloride (3×170 mL). The combined methylene chloride extracts were dried over sodium sulfate, filtered, and concentrated on a rotary evaporator to afford 8.46 g as an oil. Anal. Found: C, 62.78; H, 12.21; N, 17.84.

### Ex. 50

To a stirred mixture of DAB-8 (4 g), methylene chloride (250 mL), and sodium bicarbonate (14.5 g) was added acetyl chloride (3.57 g). After stirring overnight, a solid formed. The methylene chloride layer was decanted, and the solid residue was taken up in deionized water (300 mL), and 50% NaOH was added until pH 13. This solution was washed with methylene chloride (3×200 mL). The aqueous layer was concentrated on a rotary evaporator and precipitated with the addition of methanol. This solution was filtered and concentrated on a rotary evaporator. To the residue was added methanol (100 mL) and the mixture was stirred for 32 h, and filtered. The filtered solution was concentrated on a rotary evaporator. The residue was diluted with methylene chloride (130 mL). This mixture was filtered and the filtered solution was concentrated on a rotary evaporator. The residue was again diluted with methylene chloride (250 mL). This mixture was filtered and the filtered solution was concentrated on a rotary evaporator to afford 9.3 g.

### Ex. 51

Concentrated HCl (104.10 g) was added over a period of 90 min to DAB-4 (167.2 g, 0.528 mol) that was cooled in an ice-water bath, keeping the temperature of the DAB-4 solution less than 10 degrees C. In a separate flask dodecylbenzenesulfonic acid, sodium salt (30.8 g) and deionized water (71.87 g) was heated at 60 degrees C. for 20 min until all was dissolved. The dodecylbenzenesulfonic acid, sodium salt solution was added to the DAB-4 solution. To the resulting solution, under a nitrogen atmosphere, was added toluene (1086 g) and the mixture was heated to 80 degrees C. With rapid stirring, a solution of EPI (103.25 mL, 122.14 g, 1.32 mol) in toluene (155 mL) was added dropwise over a period of 2 h. After the addition, the reaction was heated further at 80 degrees C. for 6 h and allowed to cool to room temperature. The reaction mixture was filtered. The collected solid was suspended in methanol (2 L), stirred 20 min, and filtered. This methanol was repeated twice more. The filtered solid was suspended in 20% aqueous NaOH (2 L), stirred 20 min, and filtered. This 20% aqueous NaOH wash was repeated twice more. The filtered solid was suspended in methanol (2 L), stirred 20 min, and filtered. This methanol was repeated once more. The filtered solid was suspended in deionized water (4 L), stirred 20 min, and filtered. This deionized water wash was repeated twice more. The filtered polymer (wet weight 759.54 g) was lyophilized to afford 192.66 g. In-process-swelling ration was 3.15 mL/g. In vitro phosphate binding was 0.86 and 0.74 mmol/g, at 1 h and 5 h, respectively.

### Ex. 52

Concentrated HCl (12.5 mL) was added over a period of 90 min to DAB-4 (20 g, 0.0632 mol) that was cooled in an ice-water bath, keeping the temperature of the DAB-4 solution less than 10 degrees C. In a separate flask dodecylbenzenesulfonic acid, sodium salt (3.69 g) and deionized water (8.60 mL) was stirred until all was dissolved. The dodecylbenzenesulfonic acid, sodium salt solution was added to the DAB-4 solution. To the resulting solution, under a nitrogen atmosphere, was added toluene (150 mL) and the mixture was heated to 80 degrees C. With rapid stirring, a solution of EPI (24.72 mL, 29.24 g, 0.316 mol) in toluene (19 mL) was added dropwise over a period of 2 h. After the addition, the reaction was heated further at 80 degrees C. for 6 h and allowed to cool to room temperature. The reaction mixture was filtered. The collected solid was suspended in methanol (1 L), stirred 20 min, and filtered. This methanol was repeated twice more. The filtered solid was suspended in 20% aqueous NaOH (1 L), stirred 20 min, and filtered. This 20% aqueous NaOH wash was repeated twice more. The filtered solid was suspended in methanol (1 L), stirred 20 min, and filtered. This methanol was repeated once more. The filtered solid was suspended in deionized water (2 L), stirred 20 min, and filtered. This deionized water wash was repeated twice more. The filtered polymer (wet weight 51.81 g) was lyophilized to afford 26.61 g. In-process-swelling ratio was 0.947 mL/g.

### Ex. 53

Concentrated HCl (12.5 mL) was added over a period of 90 min to DAB-4 (20 g, 0.0632 mol) that was cooled in an ice-water bath, keeping the temperature of the DAB-4 solution less than 10 degrees C. In a separate flask dodecylbenzenesulfonic acid, sodium salt (3.69 g) and deionized water (8.60 g) was stirred until all was dissolved. The dodecylbenzenesulfonic acid, sodium salt solution was added to the DAB-4 solution. To the resulting solution, under a nitrogen atmosphere, was added toluene (150 mL) and the mixture was heated to 80 degrees C. With rapid stirring, a solution of EPI (4.94 mL, 5.84 g, 0.0632 mol) in toluene (19 mL) was added dropwise over a period of 2 h. After the addition, the reaction was heated further at 80 degrees C. for 6 h and allowed to cool to room temperature. The reaction mixture was filtered. The collected solid was suspended in methanol (2 L), stirred 20 min, and filtered. This methanol was repeated twice more. The filtered solid was suspended in 20% aqueous NaOH (2 L), stirred 20 min, and filtered. This 20% aqueous NaOH wash was repeated twice more. The filtered solid was suspended in methanol (2 L), stirred 20 min, and filtered. This methanol was repeated once more. The filtered solid was suspended in deionized water (4 L), stirred 20 min, and filtered. This deionized water wash was repeated twice more. The filtered polymer (wet weight 144.57 g) was lyophilized. The lyophilized material was suspended in deionized water, and concentrated HCl was added to the suspension until pH 5. Lyophilization afforded 20.38 g. In-process-swelling ratio was 6.09 mL/g.

### Ex. 54

Concentrated HCl (104.10 g) was added over a period of 90 min to DAB-4 (167.2 g, 0.528 mol) that was cooled in an ice-water bath, keeping the temperature of the DAB-4 solution less than 10 degrees C. In a separate flask dodecylbenzenesulfonic acid, sodium salt (30.8 g) and deionized water (71.87 g) was heated at 60 degrees C. for 20 min until all was dissolved. The dodecylbenzenesulfonic acid, sodium salt solution was added to the DAB-4 solution. To the resulting solution, under a nitrogen atmosphere, was added toluene (1086 g) and the mixture was heated to 80 degrees C. With rapid stirring, a solution of EPI (103.25 mL, 122.14 g, 1.32 mol) in toluene (155 mL) was added dropwise over a period of 2 h. After the addition, the reaction was heated further at 80 degrees C. for 6 h and allowed to cool to room temperature. The reaction mixture was filtered. The collected solid was suspended in methanol (2 L), stirred 20 min, and filtered. This methanol was repeated twice more. The filtered solid was suspended in 20% aqueous NaOH (2 L), stirred 20 min, and filtered. This 20% aqueous NaOH wash was repeated twice more. The filtered solid was suspended in methanol (2 L), stirred 20 min, and filtered. This methanol was repeated once more. The filtered solid was suspended in deionized water (4 L), stirred 20 min, and filtered. This deionized water wash was repeated twice more. The filtered polymer (wet weight 560.24 g) was lyophilized to afford 139.71 g. In-process-swelling ratio was 3.01 mL/g. Anal. Found: C, 59.77; H, 11.16; N, 17.67; Cl, 1.41; S, <0.11.

### Ex. 55

To a stirred solution of DAB-4 (25 g) and deionized water (16.14 g) at room temperature was added EPI (2.92 g). The reaction temperature rose to 63 degrees C., during the addition. After the addition was complete, the solution was heated to 80 degrees C. for 18 h. No gel formed. The reaction was heated to 90 degrees C. for 2 h and allowed to cool to room temperature. No gel formed. To the reaction was added another portion of EPI (15.40 g). The reaction temperature rose to 74 degrees C. and the reaction gelled. The reaction was heated to 80 degrees C. for 18 h and 90 degrees C. for 2 h. After cooling to room temperature, the gel was broken into small pieces and suspended in 4 L deionized water, stirred, and filtered. This wash was repeated once more. The filtered gel was resuspended in 4 L deionized water and stirred (conductivity of suspension 0.24 mS/cm). The washed polymer (wet weight 205.81 g) was dried in a forced-air oven at 60 degrees C. to afford 27.01 g. This dried polymer was suspended in deionized water (3 L) and stirred for 1 h (suspension pH 9.7). Concentrated HCl was added to this suspension until pH 5, and the suspension was filtered. The washed polymer (wet weight 255.73 g) was dried in a forced-air oven at 60 degrees C. to afford 36.13 g. In-process-swelling 6.08 mL/g.

### Ex. 56

A solution of 2.2 g (13 mmol) of 4-vinylbenzyl chloride (technical grade 90%, commercially available from Aldrich) in 20 ml of chloroform was added to the stirred mixture of 10 g (13 mmol) of DAB-8, 2.69 g (19.5 mmol) of potassium carbonate anhydrous in 500 ml of chloroform for 1 hour. After stirring overnight at room temperature the mixture was filtered. The filtrate was collected, dried over potassium carbonate and concentrated on a rotary evaporator to give 11.4 g of product as a yellow oil.

### Ex. 57

11.4 g of 4-vinylbenzyl chloride modified DAB-8 (Ex. 56) was added to a 250 ml 3-necked flask. The flask was equipped with an overhead stirrer, 25 ml addition funnel, thermocouple, pH meter. 34 ml of deionized water was added. The mixture was stirred and cooled to 7 degrees C. with an ice bath. 37% HCl was added via addition funnel dropwise until pH 1 maintaining a temperature between 7-15 degrees C. The cooling bath was then removed and the mixture was purged with nitrogen for 20 min. 2,2′-Azobis(2-amidinopropane) dihydrochloride (114 mg) was added, the mixture was purged with nitrogen for another 10 min. The flask was connected to a nitrogen line. The reaction mixture was stirred at 55 degrees C. for 4.5 hours. The mixture was left overnight at room temperature. Gel formation was observed. The gel was placed in 2 L beaker, added 1 L of deionized water, stirred for 30 min. Most of the gel was dissolved. The mixture was cooled to 10 degrees C. with an ice bath. 50% Solution of sodium hydroxide in water was added via addition funnel dropwise until pH 10.1. The temperature was maintained between 10-20 degrees C. The solution was concentrated on a rotary evaporator to 400 ml volume. The mixture was dialyzed against deionized water (membrane molecular weight cut-off: 3,500) and lyophilized to afford: 11.0 g. The lyophilized material was placed suspended in 700 ml deionized water and the mixture was stirred for 30 min (suspension pH 10.0). Concentrated HCl was added until suspension pH 7.5. The suspension was filtered, and the wet polymer (wet weight 107.2 g) was lyophilized to afford 7.4 g.

### Ex. 58

30 g of 4-vinylbenzyl chloride modified DAB-8 (Ex. 56) was added to a 250 ml 3-necked flask. The flask was equipped with an overhead stirrer, 25 ml addition funnel, thermocouple, pH meter. 90 ml of deionized water was added. The mixture was stirred and cooled to 7 degrees C. with an ice bath. Concentrated HCl was added via addition funnel dropwise until pH 1.0, maintaining a temperature between 7-15 degrees C. The cooling bath was then removed and the mixture was purged with nitrogen for 20 min. 2,2′-Azobis(2-amidinopropane) dihydrochloride (300 mg) was added, the mixture was purged with nitrogen for another 10 min. The flask was connected to a nitrogen line. The reaction mixture was stirred at 55 degrees C. for 3 hours. NMR 1H was taken. NMR indicates disappearance of vinyl protons. The mixture was cooled to 10 degrees C. with an ice bath. 50% Solution of sodium hydroxide in water was added via addition funnel dropwise until pH 10.5. The mixture was dialyzed against deionized water (MWCO: 3,500) and lyophilized to afford 17.56 g.

### Ex. 59

Poly{N-(DAB-8)methyl vinylbenzene} (10.0 g, Ex. 58) was placed in 100 ml 3 necked flask equipped with overhead stirrer. 35 ml of deionized water was added. Mixture was stirred for 2 hours until the polymer dissolved. 0.77 g (8.4 mmol) of EPI was added. The mixture was stirred for 4 hours. Gel formation was observed after 2 hours. The mixture was left overnight at room temperature. The gel was placed in 2 L beaker, added 1.4 L of deionized water, stirred for 30 min (conductivity 0.94 mS/cm, pH 9.1). Several drops of concentrated HCl were added until pH 7.2. Gel was filtered off (wet weight 152 g). Gel was dried in forced air oven (60 degrees C.) for 20 hours to afford 7.1 g.

### Ex. 60

7.2 g of poly{N-(DAB-8)methyl vinylbenzene} (Ex. 58) was placed in 100 ml 3 necked flask, equipped with overhead stirrer. 25 ml of deionized water was added. Mixture was stirred for 1.5 hours until the polymer dissolved. 1.11 g (12 mmol) of EPI was added. The mixture was stirred for 3.5 hours. Gel formation was observed after 1.5 hours. The mixture was left overnight at room temperature. The gel was placed in 2 L beaker, 1.4 L of deionized water was added, the mixture was stirred for 30 min (conductivity 0.87 mS/cm, pH 9.0). Several drops of concentrated HCl were added until pH 7.7. Gel was filtered off (wet weight 164 g). Gel was dried in forced air oven (60 degrees C.) for 20 hours to afford 5.3 g.

### Ex. 61

A solution of 1.51 g (8.9 mmol) 4-vinylbenzyl chloride (technical grade 90%, commercially available from Aldrich) in 20 ml of chloroform was added to the stirred mixture of 15 g (8.9 mmol) of DAB-16, 2.53 g (18.3 mmol) of potassium carbonate anhydrous in 120 ml of chloroform for 1 hour. After stirring overnight at room temperature the mixture was filtered. The filtrate was collected, dried over potassium carbonate and rotovapped to give 16.3 g of product as a yellow oil.

### Ex. 62

4-Vinylbenzyl chloride modified DAB-16 (15.5 g, Ex. 61) was added to a 250 ml 3-necked flask. The flask was equipped with an overhead stirrer, 25 ml addition funnel, thermocouple, pH meter. 60 ml of deionized water was added. The mixture was stirred and cooled to 7 degrees C. with an ice bath. Concentrated HCl was added via addition funnel dropwise until pH 1.2, maintaining a temperature between 7-15 degrees C. The cooling bath was then removed and the mixture was purged with nitrogen for 15 min. 2,2′-Azobis(2-amidinopropane) dihydrochloride (155 mg) was added, the mixture was purged with nitrogen for another 15 min. The flask was connected to a nitrogen line. The reaction mixture was stirred at 55 degrees C. for 3.5 h. 1H NMR indicated disappearance of vinyl protons. The mixture was allowed to return to room temperature. The mixture was cooled to 10 degrees C. with an ice bath. NaOH (50% aqueous solution) was added via addition funnel dropwise until pH 10.5. Temperature was maintained between 10-20 degrees C. The solution was concentrated on a rotary evaporator (45 g less). EPI (1.43 g, 15.5 mmol) of EPI was added. The mixture was stirred at room temperature for 1.5 hours and at 55 degrees C. for another 3.5 hours. After 10 min at 55 degrees C. a gel formed. The mixture was left overnight at room temperature. The gel was placed in 5 L beaker, added 2.5 L of deionized water, stirred for 30 min. Gel was filtered off, placed back in the beaker, added 2.5 L of deionized water, stirred for 30 min (conductivity 0.96 mS/cm, pH 8.6). Several drops of concentrated HCl were added until pH 7.3. Gel was filtered off (wet weight 207 g) and lyophilized to afford 12.6 g.

### Ex. 63

To a solution of DAB-16 in deionized water cooled in an ice water bath was added concentrated HCl (6.28 g). The solution had pH 7. The solution contains the equivalent of 22.88% (w/w) of DAB-16.

### Ex. 64

To a stirred solution of DAB-16 (10.33 g) in deionized water (40 ml) was added concentrated HCl (9.5 ml) until the solution had pH 8.1. Lyophilization afforded 11.9 g.

### Ex. 65

A solution of 4-vinylbenzyl chloride (28 g, technical grade 90%, commercially available from Aldrich) in 30 ml of chloroform was added over a period of 4 h to a stirred mixture of DAB-Am-4 (272 mL), anhydrous potassium carbonate (30.3 g), and chloroform (1300 mL). After stirring overnight at room temperature the mixture was filtered. The filtrate was extracted twice with borate buffer (1.3 L each extraction; borate buffer prepared by mixing 115.7 g boric acid, 37.44 g NaOH, and 2.6 L deionized water; buffer pH 9.5). The aqueous layers were combined and NaOH (40% aqueous solution) was added until pH 12.4. The aqueous layer was extracted twice with chloroform (1.4 L each). The combined chloroform extracts were dried over potassium carbonate, filtered and concentrated on a rotary evaporator to give 38.1 g of a yellow oil.

### Ex. 66

To a solution of vinylbenzylchloride modified DAB-Am-4 (17 g, Ex. 65), in deionized water (58.56 g) was added concentrated HCl until the solution had pH 1.0. The solution was put under a nitrogen atmosphere, then 2,2′-azobis(2-amidinopropane) dihydrochloride (170 mg) was added and the solution was heated at 55 degrees C. overnight. After cooling to room temperature NaOH (50% aqueous solution) was added until the solution had pH 11. Epichlorohydrin (0.32 g) was added with stirring. A gel formed within 35 min. After curing at room temperature for 4 days, the gel was broken into small pieces and suspended in deionized water (3 L), stirred, and filtered. The filtered polymer was suspended in deionized water (2.5 L), stirred, and filtered. The filtered polymer was suspended in deionized water (3 L) and stirred (conductivity 400 uS/cm, pH 9.6). Concentrated HCl was added to the stirred suspension until pH 7.9, and the suspension was filtered. The filtered material (wet weight 1228 g) was dried in a forced-air oven at 60 degrees C. to afford 12.7 g.

### Ex. 67

A solution of 4-vinylbenzyl chloride (23.8 g, technical grade 90%, commercially available from Aldrich) in 30 ml of chloroform was added over a period of 3 h to a stirred mixture of DAB-Am-4 (177.6 g), potassium carbonate anhydrous (25.73 g), and chloroform (1100 mL). After stirring over 3 nights at room temperature the mixture was filtered. The filtrate was concentrated on a rotary evaporator to 650 mL, and extracted twice with borate buffer (1.25 L each extraction; borate buffer preparation by mixing 105.18 g boric acid, 34 g NaOH, and 2.5 L deionized water). The aqueous layers were combined and NaOH (50% aqueous solution) was added until pH 12.4. The aqueous layer was extracted twice with chloroform (1.3 L each). The combined chloroform extracts were dried over potassium carbonate, filtered and concentrated on a rotary evaporator to give 42.6 g.

### Ex. 68

To a solution of vinylbenzylchloride modified DAB-Am-4 (21.6 g, Ex. 75), in deionized water (50.5 mL), cooled in an ice-water bath, was added concentrated HCl until the solution had pH 1.1. The solution was put under a nitrogen atmosphere, then 2,2′-azobis(2-amidinopropane) dihydrochloride (170 mg) was added and the solution was heated at 55 degrees C. for 3 h. After cooling to room temperature NaOH (50% aqueous solution) was added until the solution had pH 10.45. The solution was diluted with deionized water (20 mL). Four portions (32.65 g) of this solution were portioned out.

### Ex. 69

To one portion of this solution (32.65 g, Ex. 68) was added epichlorohydrin (0.173 g) with stirring at room temperature. A gel formed in 29 min. After curing overnight at room temperature the gel was suspended in deionized water (2 L), stirred, and filtered. The filtered polymer was suspended in deionized water (1.7 L) and stirred (conductivity 0.92 mS/cm, pH9.1). Concentrated HCl was added until the suspension had pH 8.2 and the suspension was stirred and filtered (wet weight 127.45 g). The material was dried in a forced-air oven at 60 degrees C. to afford 4.5 g.

### Ex. 70

To one portion of this solution (32.65 g, Ex. 68) was added epichlorohydrin (0.346 g) with stirring at room temperature. A gel formed in 28 min. After curing overnight at room temperature the gel was suspended in deionized water (2 L), stirred, and filtered. The filtered polymer was suspended in deionized water (1.7 L) and stirred (conductivity 0.84 mS/cm, pH 9.1). Concentrated HCl was added until the suspension had pH 8.3 and the suspension was stirred and filtered (wet weight 106.39 g). The material was dried in a forced-air oven at 60 degrees C. to afford 4.6 g.

### Ex. 71

To one portion of this solution (32.65 g, Ex. 68) was added epichlorohydrin (0.519 g) with stirring at room temperature. A gel formed in 17 min. After curing overnight at room temperature the gel was suspended in deionized water (2 L), stirred, and filtered. The filtered polymer was suspended in deionized water (1.7 L) and stirred (conductivity 0.63 mS/cm, pH 8.6). Concentrated HCl was added until the suspension had pH 8.0 and the suspension was stirred and filtered (wet weight 117.1 g). The material was dried in a forced-air oven at 60 degrees C. to afford 4.8 g.

### Ex. 72

To one portion of this solution (32.65 g, Ex. 68) was added epichlorohydrin (0.692 g) with stirring at room temperature. A gel formed in 15 min. After curing overnight at room temperature the gel was suspended in deionized water (2 L), stirred, and filtered. The filtered polymer was suspended in deionized water (1.7 L) and stirred (conductivity 0.58 mS/cm, pH 8.5). Concentrated HCl was added until the suspension had pH 7.8 and the suspension was stirred and filtered (wet weight 106.8 g). The material was dried in a forced-air oven at 60 degrees C. to afford 4.9 g.

### Ex. 73

A solution of 4-vinylbenzyl chloride (19.28 g, technical grade 90%, commercially available from Aldrich) in 30 ml of chloroform was added over a period of 3 h to a stirred mixture of DAB-Am-4 (144 g), potassium carbonate anhydrous (20.8 g), and chloroform (700 mL). After stirring overnight at room temperature the mixture was filtered. The filtrate was extracted twice with borate buffer (700 mL each extraction; borate buffer preparation by mixing 62.5 g boric acid, 8 g NaOH, and 1.4 L deionized water). The aqueous layers were combined and NaOH (50% aqueous solution) was added until pH 12.7. The aqueous layer was extracted twice with chloroform (1 L each). The combined chloroform extracts were dried over potassium carbonate, filtered and concentrated on a rotary evaporator to give 31.8 g of a yellow oil.

### Ex. 74

To a solution of 4-vinylbenzylchloride modified DAB-Am-4 (10 g, Ex. 73), in deionized water (14 mL), cooled in an ice-water bath, was added concentrated HCl until the solution had pH 1. The solution was put under a nitrogen atmosphere, then N,N′-ethylenebisacrylamide (0.375 g) and 2,2′-azobis(2-amidinopropane) dihydrochloride (100 mg) were added. This solution was added via syringe to a solution of poly(vinyl acetate) (10 g) in toluene (300 mL) under a nitrogen atmosphere. With vigorous stirring the mixture was heated to 65 degrees C. for 2 h 20 min. After cooling to room temperature the mixture was filtered. The filtered material was suspended in methanol (800 mL), stirred, and filtered. The filtered material was suspended in methanol (800 mL), stirred, and filtered. The filtered material was suspended in deionized water (1.5 L) and stirred (conductivity 0.62 mS/cm, pH 3.6). NaOH (50% aqueous solution) was added to the suspension until pH 7.2. The material was filtered (wet weight 228 g) and dried in a forced-air oven at 60 degrees C. to afford 11.9 g.

### Ex. 75

A solution of poly(epichlorohydrin) (1.04 g, commercially available from Aldrich), DAP-Am-4 (10.72 g), and 1-methyl-2-pyrrolidinone (80 mL) was heated at 140 degrees C. for 48 h. After cooling to room temperature the reaction solution was poured into ether and after standing overnight, the liquid layer was decanted from the precipitate. The precipitate was dissolved in deionized water and dialyzed against deionized water (membrane MWCO 3500). The dialyzed solution was concentrated in a forced-air oven at 60 degrees C., and lyophilized to afford 1.45 g.

### Ex. 76

A portion of DAP-Am-4 modified poly(epichlorohydrin) (1.37 g, Ex. 75) was suspended in deionized water (12 g) and a few drops of NaOH (50% aqueous solution) and heated in a sealed container at 60 degrees C. After cooling to room temperature the mixture was diluted with deionized water (6 g) and the suspension was adjusted to pH 10. The solution was put under a nitrogen atmosphere then epichlorohydrin (30 uL) was added and after stirring 4 h at room temperature another portion of epichlorohydrin (30 uL) was added and after stirring overnight at room temperature a third portion of epichlorohydrin (30 uL) was added. After heating overnight at 60 degrees C. then cooling to room temperature, the mixture was suspended into deionized water (250 mL). The suspension was adjusted to pH 7. After stirring for 1 h at room temperature the mixture was dialyzed against deionized water. The dialyzed solution was dried in a forced-air oven at 60 degrees C. to afford 1.35 g. In vitro phosphate binding was 0.00 and 0.00 mmol/g, at 1 h and 5 h, respectively.

### Ex. 77

A solution of 4-vinylbenzyl chloride (0.405 mL, technical grade 90%, commercially available from Aldrich) in 20 ml of chloroform was added over a period of 55 min to a stirred mixture of DAB-Am-8 (2 g), potassium carbonate anhydrous (0.538 g), and chloroform (100 mL). After stirring overnight at room temperature the mixture was filtered. The filtrate was collected and concentrated on a rotary evaporator to give 2.35 g.

### Ex. 78

To a solution of DAB-Am-8 reacted with 4-vinylbenzylchloride (2.3 g, Ex. 77), in deionized water (7 mL) was slowly added concentrated HCl until the solution had pH 1.0. The solution was put under a nitrogen atmosphere, then 2,2′-azobis(2-amidinopropane) dihydrochloride (23 mg) was added and the solution was heated at 55 degrees C. for 3 h 40 min, followed by 60 degrees C. for 2 h. After cooling to room temperature deionized water (150 mL) was added with stirring. NaOH (50% aqueous solution) was added until the solution had pH 10.9. The mixture was diluted with deionized water (50 mL), dialyzed against deionized water (membrane MWCO 3500), and lyophilized to afford 1.04 g. Deionized water (104 mL) was added to the lyophilized material and concentrated HCl was added until pH 8.1. The mixture was filtered and the filtered material (wet weight 12.8 g) was dried in a forced-air oven at 60 degrees C. to afford 0.90 g. In vitro phosphate binding was 0.51 and 0.19 mmol/g, at 1 h and 5 h, respectively.

### Ex. 79

A solution of 4-vinylbenzyl chloride (0.248 g, technical grade 90%, commercially available from Aldrich) in 30 ml of chloroform was added over a period of 1 h to a stirred mixture of DAB-Am-16 (2.8 g), potassium carbonate anhydrous (0.338 g), and chloroform (100 mL). After stirring overnight at room temperature the mixture was filtered. The filtrate was collected and concentrated on a rotary evaporator to give 2.85 g.

### Ex. 80

To a solution of DAB-Am-16 reacted with 4-vinylbenzylchloride (2.81 g, Ex. 79), in deionized water (8.9 mL) was slowly added concentrated HCl until the solution had pH 1.1. The solution was put under a nitrogen atmosphere, then 2,2′-azobis(2-amidinopropane) dihydrochloride (28 mg) was added and the solution was heated at 55 degrees C. for 3 h. After cooling to room temperature the reaction mixture was dialyzed against deionized water (membrane MWCO 3500), and lyophilized to afford 2.78 g.

### Ex. 81

To a mixture of polymerized DAB-Am-16 reacted with 4-vinylbenzylchloride (2.65 g, Ex. 80), and deionized water (10 mL), cooled in an ice-water bath, was added NaOH (50% aqueous solution) until pH 10.3. Epichlorohydrin (0.0205 g) was added and the mixture was stirred at room temperature for 6 h followed by 60 degrees C. overnight. Another portion of epichlorohydrin (0.222 g) was added the mixture was stirred 30 min until a gel formed. After curing at room temperature for 16 h the gel was broken into small pieces and, suspended in deionized water (600 mL), stirred, and filtered. The filtered material was suspended in deionized water (600 mL) and stirred (conductivity 0.27 mS/cm, pH 8.5). Concentrated HCl was added until suspension pH 7.7. Filtration and lyophilization afforded 1.56 g. In vitro phosphate binding was 0.27 and 0.11 mmol/g, at 1 h and 5 h, respectively.

### Ex. 82

A solution of poly(epichlorohydrin) (3.5 g), DAB-Am-4 (27.23 g), and 1-methyl-2-pyrrolidinone (240 mL) was heated at 140 degrees C. for 48 h. After cooling to room temperature the reaction solution was poured into ether and after standing overnight, the liquid layer was decanted from the precipitate. The precipitate was dissolved in deionized water and dialyzed against deionized water (membrane MWCO 3500). The dialyzed solution was concentrated in a forced-air oven at 60 degrees C., and lyophilized to afford 7.8 g. A portion of this material (7.5 g) was suspended in deionized water (60 g) and a few drops of NaOH (50% aqueous solution) and heated in a sealed container at 60 degrees C. After cooling to room temperature the mixture was diluted with deionized water and the suspension was adjusted to pH 10. After stirring for 1 h the mixture was filtered and dried overnight in a forced-air oven at 60 degrees C. Deionized water was added to the dried material and the stirred suspension was adjusted to pH 7. After stirring for 1 h, the mixture was filtered. The filtered material was suspended in deionized water (1 L), stirred 30 min, and filtered. The filtered material was dried in a forced-air oven at 60 degrees C. to afford 7.12 g. In vitro phosphate binding was 0.00 and 0.00 mmol/g, at 1 h and 5 h, respectively.

### Results: Amine Polymer Urinary Phosphate Reduction (In Vivo-Rats)

The following tables provide in vivo sequestration data for urinary phosphate reduction in rats, in accordance with the methodology set forth below. The example numbers provided in the tables refer to the examples presented above.

TABLE 11

| | Polymer Dose | Urinary | % Reduction |
|---|---|---|---|
| | (% weight of | Phosphorous | of Urinary |
| Ex. No. | feed) | (mg/day) | Phosphorous |
| Negative | 0.50 | 22.9 | NA |
| Control | | | |
| Positive | 0.50 | 12.7 | 44.4 |
| Control | | | |
| 12 | 0.50 | 8.0 | 65.1 |
| 13 | 0.50 | 7.7 | 66.4 |

TABLE 12

| | Polymer Dose | Urinary | % Reduction |
|---|---|---|---|
| | (% weight of | Phosphorous | of Urinary |
| Ex. No. | feed) | (mg/day) | Phosphorous |
| Negative | 0.50 | 15.8 | NA |
| Control | | | |
| Positive | 0.50 | 9.1 | 42.7 |
| Control | | | |
| 41 | 0.50 | 8.3 | 47.7 |
| 49 | 0.50 | 8.9 | 43.5 |

TABLE 13

| | Polymer Dose | Urinary | % Reduction |
|---|---|---|---|
| | (% weight of | Phosphorous | of Urinary |
| Ex. No. | feed) | (mg/day) | Phosphorous |
| Negative | 0.50 | 19.6 | NA |
| Control | | | |
| Positive | 0.50 | 9.4 | 52.2 |
| Control | | | |
| 15 | 0.50 | 7.1 | 63.7 |
| 40 | 0.50 | 17.5 | 10.7 |
| 42 | 0.50 | 7.2 | 63.2 |
| 44 | 0.50 | 6.1 | 69.0 |
| 44 | 0.35 | 11.6 | 41.2 |
| 44 | 0.25 | 11.0 | 44.2 |

TABLE 14

| | Polymer Dose | Urinary | % Reduction |
|---|---|---|---|
| | (% weight of | Phosphorous | of Urinary |
| Ex. No. | feed) | (mg/day) | Phosphorous |
| Negative | 0.50 | 23.3 | NA |
| Control | | | |
| Positive | 0.50 | 14.4 | 38.1 |
| Control | | | |
| Positive | 1.00 | 8.7 | 62.7 |
| Control | | | |
| 18 | 0.50 | 16.8 | 27.7 |
| 19 | 0.50 | 16.2 | 30.5 |
| 22 | 0.50 | 13.2 | 43.1 |
| 23 | 0.50 | 14.2 | 39.0 |
| 24 | 0.50 | 11.2 | 51.9 |
| 25 | 0.50 | 11.3 | 51.3 |
| 31 | 0.50 | 8.8 | 62.4 |
| 59 | 0.50 | 15.6 | 67.1 |

TABLE 15

| | Polymer Dose | Urinary | % Reduction |
|---|---|---|---|
| | (% weight of | Phosphorous | of Urinary |
| Ex. No. | feed) | (mg/day) | Phosphorous |
| Negative | 0.50 | 17.2 | NA |
| Control | | | |
| Positive | 0.50 | 9.3 | 45.6 |
| Control | | | |
| 20 | 0.50 | 6.2 | 63.8 |
| 21 | 0.50 | 7.5 | 56.4 |
| 31 | 0.50 | 6.9 | 59.9 |
| 32 | 0.50 | 10.9 | 36.6 |
| 61 | 0.50 | 8.9 | 48.4 |
| 62 | 0.50 | 9.8 | 43.1 |

TABLE 16

| | Polymer Dose | Urinary | % Reduction |
|---|---|---|---|
| | (% weight of | Phosphorous | of Urinary |
| Ex. No. | feed) | (mg/day) | Phosphorous |
| Negative | 0.50 | 15.1 | NA |
| Control | | | |
| Positive | 0.50 | 9.6 | 36.5 |
| Control | | | |
| 7 | 0.50 | 13.1 | 13.0 |
| 10 | 0.50 | 14.5 | 3.8 |
| 56 | 0.50 | 10.8 | 28.6 |
| 69 | 0.49 | 8.6 | 42.7 |
| 70 | 0.50 | 10.8 | 28.4 |
| 71 | 0.50 | 9.9 | 34.6 |
| 72 | 0.50 | 8.4 | 44.7 |

TABLE 17

| | Polymer Dose | Urinary | % Reduction |
|---|---|---|---|
| | (% weight of | Phosphorous | of Urinary |
| Ex. No. | feed) | (mg/day) | Phosphorous |
| Negative | 0.50 | 15.6 | NA |
| Control | | | |
| Positive | 0.50 | 8.1 | 48.0 |
| Control | | | |
| 74 | 0.50 | 11.8 | 24.7 |

TABLE 18

| | Polymer Dose | Urinary | % Reduction |
|---|---|---|---|
| | (% weight of | Phosphorous | of Urinary |
| Ex. No. | feed) | (mg/day) | Phosphorous |
| Negative | 0.50 | 18.4 | NA |
| Control | | | |
| Positive | 0.50 | 8.2 | 55.2 |
| Control | | | |
| 54 | 0.50 | 15.9 | 13.5 |
| 55 | 0.50 | 11.1 | 39.5 |
| 56 | 0.50 | 7.3 | 60.5 |

TABLE 19
[Effect of High Fat Diet on Phosphate Binding]

| | Polymer Dose | Urinary | % Reduction |
|---|---|---|---|
| | (% weight of | Phosphorous | of Urinary |
| Ex. No. | feed) | (mg/day) | Phosphorous |
| Negative | 0.50 | 13.4 | NA |
| Control | | | |
| Positive | 0.25 | 8.0 | 40.5 |
| Control | | | |
| Positive | 0.50 | 6.7 | 50.2 |
| Control | | | |
| Positive | 1.00 | 3.3 | 75.3 |
| Control | | | |
| 14 | 0.50 | 3.5 | 73.9 |
| 56 | 0.50 | 7.2 | 46.3 |

TABLE 20

| | Polymer Dose | Urinary | % Reduction |
|---|---|---|---|
| | (% weight of | Phosphorous | of Urinary |
| Ex. No. | feed) | (mg/day) | Phosphorous |
| Negative | 0.50 | 20.0 | NA |
| Control | | | |
| Positive | 0.50 | 12.1 | 39.4 |
| Control | | | |
| 16 | 0.50 | 9.6 | 51.9 |
| 16 | 0.25 | 15.8 | 21.2 |
| 17 | 0.50 | 9.6 | 51.8 |
| 17 | 0.25 | 12.4 | 37.9 |
| 43 | 0.50 | 11.0 | 45.2 |
| 56 | 0.50 | 13.6 | 32.2 |

TABLE 21

| | Polymer Dose | Urinary | % Reduction |
|---|---|---|---|
| | (% weight of | Phosphorous | of Urinary |
| Ex. No. | feed) | (mg/day) | Phosphorous |
| Negative | 0.50 | 15.3 | NA |
| Control | | | |
| Positive | 0.50 | 8.2 | 46.8 |
| Control | | | |
| Positive | 1.0 | 4.0 | 73.6 |
| Control | | | |
| 26 | 0.50 | 4.3 | 71.9 |

TABLE 22

| | Polymer Dose | Urinary | % Reduction |
|---|---|---|---|
| | (% weight of | Phosphorous | of Urinary |
| Ex. No. | feed) | (mg/day) | Phosphorous |
| Negative | 0.50 | 23.1 | NA |
| Control | | | |
| Positive | 0.50 | 9.7 | 58.0 |
| Control | | | |
| Positive | 1.00 | 5.5 | 76.3 |
| Control | | | |
| 26 | 0.50 | 7.7 | 66.6 |
| 53 | 0.50 | 9.0 | 61. |

TABLE 23

| | Polymer Dose | Urinary | % Reduction |
|---|---|---|---|
| | (% weight of | Phosphorous | of Urinary |
| Ex. No. | feed) | (mg/day) | Phosphorous |
| Negative | 0.50 | 15.9 | NA |
| Control | | | |
| Positive | 0.50 | 8.9 | 44.3 |
| Control | | | |
| 3 | 0.50 | 8.3 | 47.7 |

TABLE 24

| | Polymer Dose | Urinary | % Reduction |
|---|---|---|---|
| | (% weight of | Phosphorous | of Urinary |
| Ex. No. | feed) | (mg/day) | Phosphorous |
| Negative | 0.50 | 13.0 | NA |
| Control | | | |
| Positive | 0.50 | 6.6 | 48.9 |
| Control | | | |
| 66 | 0.50 | 5.7 | 55.8 |

TABLE 25

| | Polymer Dose | Urinary | % Reduction |
|---|---|---|---|
| | (% weight of | Phosphorous | of Urinary |
| Ex. No. | feed) | (mg/day) | Phosphorous |
| Negative | 0.50 | 15.1 | NA |
| Control | | | |
| Positive | 0.50 | 8.5 | 43.3 |
| Control | | | |
| 9 | 0.50 | 12.4 | 17.6 |

TABLE 26

| | Polymer Dose | Urinary | % Reduction |
|---|---|---|---|
| | (% weight of | Phosphorous | of Urinary |
| Ex. No. | feed) | (mg/day) | Phosphorous |
| Negative | 0.50 | 14.3 | NA |
| Control | | | |
| Positive | 0.50 | 7.5 | 47.2 |
| Control | | | |
| 2 | 0.50 | 10.9 | 24.0 |

TABLE 27

| | Polymer Dose | Urinary | % Reduction |
|---|---|---|---|
| | (% weight of | Phosphorous | of Urinary |
| Ex. No. | feed) | (mg/day) | Phosphorous |
| Negative | 0.50 | 17.7 | NA |
| Control | | | |
| Positive | 0.50 | 7.8 | 55.9 |
| Control | | | |
| 1 | 0.50 | 9.6 | 45.6 |

TABLE 28

| | Polymer Dose | Urinary | % Reduction |
|---|---|---|---|
| | (% weight of | Phosphorous | of Urinary |
| Ex. No. | feed) | (mg/day) | Phosphorous |
| Negative | 0.50 | 19.5 | NA |
| Control | | | |
| Positive | 0.50 | 12.6 | 35.2 |
| Control | | | |
| 63 | 2.2 (equivalent | 18.2 | 6.3 |
| | to 0.5% DAB- | | |
| | Am-16) | | |

### Methods

### Amine Polymer Urinary Phosphate Reduction (In Vivo-Rats)

House male Sprague Dawley (SD) rats were used for the experiments. The rats were placed singly in wire-bottom cages, fed with Purina 5002 diet, and allowed to acclimate for at least 5 days prior to experimental use.

To establish baseline phosphorus excretion, the rats were placed in metabolic cages for 48 hours. Their urine was collected and its phosphorus content analyzed with a Hitachi analyzer to determine phosphorus excretion in mg/day. Any rats with outlying values were excluded; and the remainder of the rats were distributed into groups.

Purina 5002 was used as the standard diet. The polymer being tested was mixed with Purina 5002 to result in a final concentration 0.25%, 0.35%, 0.5% and 1% by weight of the feed. Cellulose at 0.5% by weight was used as a negative control. Sevelamer was used as a positive control. In the event that a high-fat diet was used (see Table 19), rats were given feed comprising Purina 5002, 0.25%, 0.35%, 0.5% and 1% by weight of the feed of the polymer and 10% by weight of the feed of purified Olive oil, with the purified olive oil commercially available from Sigma. For each rat, 200 g of diet was prepared.

Each rat was weighed and placed on the standard diet. After 4 days the standard diet was replaced with the treatment or high fat diet, (or control diet for the control group). On days 5 and 6, urine samples from the rats at 24 hours (+/−30 minutes) were collected and analyzed. The test rats were again weighed, and any weight loss or gain was calculated. Any remaining food was also weighed to calculate the amount of food consumed per day. A change in phosphorus excretion relative to baseline and cellulose negative control was calculated using Excel program. Comparisons of the amounts of urinary phosphorous obtained from the test rats are shown in Tables 11-28. Percentage reduction of urinary phosphorous in a study was determined by the following equation:

% Reduction of Urinary Phosphorous=[(urinary phosphorous of negative control (mg/day)−urinary phosphorous of experimental (mg/day))/urinary phosphorous of negative control (mg/day)]×100.

In Vitro Phosphate Binding (mmol/g)

Two samples per polymer are weighed into plastic bottles after having adjusted the weight of the polymer for the loss on drying of each sample. A 10 mM phosphate buffer solution containing 10 mM KH₂PO₄, 100 mM N,N-bis[2-hydroxyethyl]-2-aminoethanesulfonic acid, 80 mM NaCl, 15 mM glycochenodeoxycholic acid (GCDC), and 15 mM oleic acid (pH was adjusted to 7.0 with 1 N NaOH) was prepared and well mixed. Aliquots of the 10 mM phosphate buffer solution were transferred into each of the two sample bottles. The solutions were well mixed and then placed into an orbital shaker at 37° C. for 1 hour. The polymer was allowed to settle prior to having removed a sample aliquot from each solution. The sample aliquot was filtered into a small vial using a disposable syringe and syringe filter. The filtered sample was diluted 1-to-10 with DI water. The shaking was continued for a further 4 hours (total of 5 hours) and the sampling procedure was repeated. Phosphate standards were prepared from a 10 mM phosphate standard stock solution and diluted appropriately to provide standards in the range of 0.3 to 1.0 mM. Both the standards and samples were analyzed by ion chromatography. A standard curve was set up and the unbound phosphate (mM) for each test solution was calculated. Bound phosphate was determined by the following equation:

Bound Phosphate (mmol/g)=[(10−Unbound PO₄)×Vol.×1000]/MassP;
wherein
   Vol.=volume of test solution (L); MassP=LOD adjusted mass of polymer (mg)
   In-Process Swelling Ratio (mL/g)

The in-process swelling ratio (SR) of several examples was determined by the following equation:

SR=(weight of wet gel (g)−weight of dry polymer (g))/weight of dry polymer (g).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A pharmaceutical composition comprising at least one polymer comprising at least one amine compound or residue thereof wherein the amine compound is represented by the following Formula I: 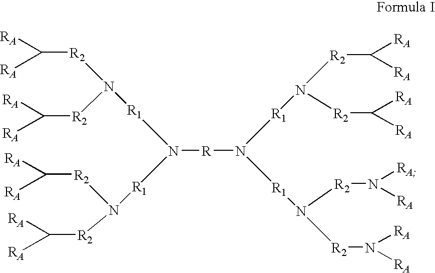
wherein
R independently represents: 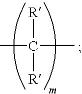
R₁ independently represents: 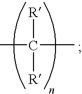
R₂ independently represents: 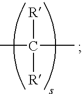
R_{A} independently represents: 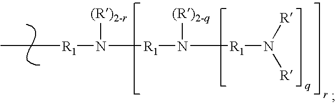
wherein m independently represents an integer from 1 to 20; n and s independently represent an integer from 1-20; q and r independently represent an integer from 0-2; and R′ independently represents a hydrogen radical; a substituted or un-substituted alkyl radical; a substituted or un-substituted aryl radical; or R′ and a neighboring R′ together represent a link or links comprising a residue of a crosslinking agent, a substituted or un-substituted alicyclic radical, a substituted or un-substituted aromatic radical, or a substituted or un-substituted heterocyclic radical; or R′ represents a link with another compound;
a crosslinking agent or residue thereof; and
a pharmaceutically acceptable excipient.

2. The composition of claim 1, wherein the crosslinking agent or residue thereof comprises epichlorohydrin or a residue thereof.

3. The composition of claim 1, wherein the crosslinking agent or residue thereof is epichlorohydrin or a residue thereof.

4. The composition of claim 1, wherein r is 0.

5. The composition of claim 1, wherein r is 2 and q is 0.

6. The composition of claim 1, wherein r is 2 and q is 2.

7. The composition of claim 1, wherein the polymer is crosslinked.

8. The composition of claim 1, wherein said another compound comprises said crosslinking agent or residue thereof or other linking compound or residue thereof wherein said other linking compound comprises amine reactive groups.

9. The composition of claim 1, wherein the polymer binds phosphate.

10. The composition of claim 9, wherein the polymer binds phosphate at greater than 0.5 mmol phosphate per gram of polymer.

11. The composition of claim 1, wherein m is 3-6, and q is 0.

12. The composition of claim 1, wherein the compound has a swelling ratio of less than 10.

13. The composition of claim 1, wherein the amine compound comprises 1,4-bis[bis[3-[bis[3-[bis(3-aminopropyl)amino]propyl]amino]propyl]amino]butane or a residue thereof.

14. The composition of claim 1, wherein the amine compound comprises 1,4-bis[bis[3-[bis[3-[bis[3-[bis(3-aminopropyl)amino]propyl]amino]propyl]amino]propyl]amino]butane or a residue thereof.

15. The composition of claim 1, wherein the amine compound comprises 1,4-bis[bis[3-[bis[3-[bis[3-[bis[3-[bis(3-aminopropyl)amino]propyl]amino]propyl]amino]propyl]amino]propyl]amino]butane or a residue thereof.

16. A method of treating hyperphosphatemia, hypocalcemia, hyperparathyroidism, depressed renal synthesis of calcitriol, tetany due to hypocalcemia, renal insufficiency, and ectopic calcification in soft tissues including calcifications in joints, lungs, kidney, conjuctiva, and myocardial tissues, chronic kidney disease, ESRD and dialysis patients comprising administering to a patient in need thereof a therapeutically effective amount of a polymer comprising at least one amine compound or residue thereof wherein the amine compound is represented by the following Formula I: 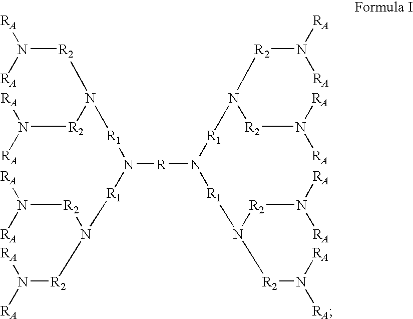
wherein
R independently represents: 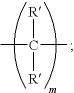
R₁ independently represents: 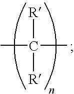
R₂ independently represents: 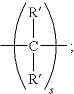
R_{A} independently represents: 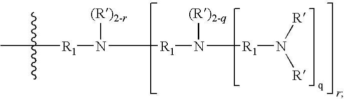
wherein m independently represents an integer from 1 to 20; n and s independently represent an integer from 1-20; q and r independently represent an integer from 0-2; and R′ independently represents a hydrogen radical; a substituted or un-substituted alkyl radical; a substituted or un-substituted aryl radical; or R′ and a neighboring R′ together represent a link or links comprising a residue of a crosslinking agent, a substituted or un-substituted alicyclic radical, a substituted or un-substituted aromatic radical, or a substituted or un-substituted heterocyclic radical; or R′ represents a link with another compound;
a crosslinking agent or residue thereof; and
a pharmaceutically acceptable excipient.

17. A polymer comprising at least one amine compound or residue thereof wherein the amine compound is represented by the following Formula I: 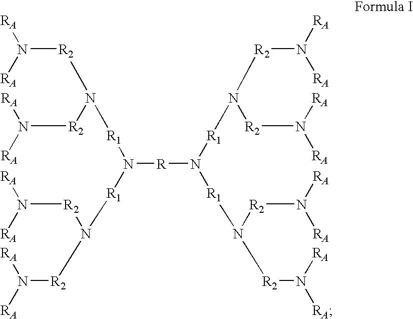
wherein
R independently represents: 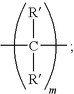
R₁ independently represents: 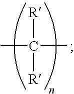
R₂ independently represents: 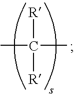
R_{A} independently represents: 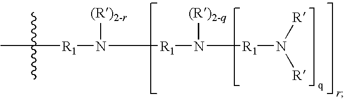
wherein m independently represents an integer from 1 to 20; n and s independently represent an integer from 1-20; q and r independently represent an integer from 0-2; and R′ independently represents a hydrogen radical; a substituted or un-substituted alkyl radical; a substituted or un-substituted aryl radical; or R′ and a neighboring R′ together represent a link or links comprising a residue of a crosslinking agent, a substituted or un-substituted alicyclic radical, a substituted or un-substituted aromatic radical, or a substituted or un-substituted heterocyclic radical; or R′ represents a link with another compound; and
a crosslinking agent or residue thereof.

18. A polymer comprising at least one amine compound or residue thereof wherein the amine compound is represented by the following Formula I: 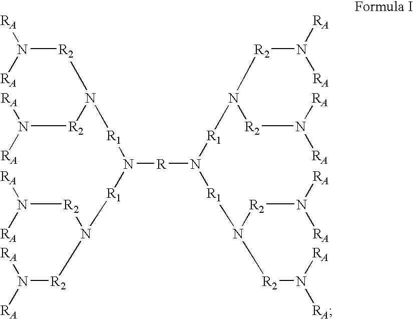
wherein
R independently represents: 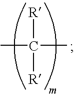
R₁ independently represents: 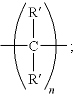
R₂ independently represents: 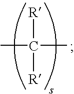
R_{A} independently represents: 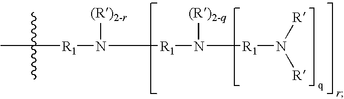
wherein m independently represents an integer from 1 to 20; n and s, independently represent an integer from 1-20; q and r independently represent an integer from 0-2; and R′ independently represents a hydrogen radical; a substituted or un-substituted alkyl radical; a substituted or un-substituted aryl radical; or R′ and a neighboring R′ together represent a link or links comprising a residue of a crosslinking agent, a substituted or un-substituted alicyclic radical, a substituted or un-substituted aromatic radical, or a substituted or un-substituted heterocyclic radical; or R′ represents a link with another compound; and
a polymerizable group or residue thereof.

19. The polymer of claim 18, wherein at least a portion of the amine compound or residue thereof is a pendant group on the polymer.

20. The polymer of claim 18, wherein the polymer is crosslinked or formed into a network.
